SEQUENCE LISTING (20 sequences)

SEQ ID NO: 1 - protein, 346 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 2 - protein, 204 aa, Homo sapiens
MNLILRYTFSKMAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFG

SEQ ID NO: 3 - protein, 115 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTP

SEQ ID NO: 4 - protein, 245 aa, Homo sapiens
MEKTELIQKAKLAEQAERYDDMATCMKAVTEQGAELSNEERNLLSVAYKNVVGGRRSAWRVISSIEQKTDTSDKKLQLIKDYREKVESELRSICTTVLELLDKYLIANATNPESKVFYLKMKGDYFRYLAEVACGDDRKQTIDNSQGAYQEAFDISKKEMQPTHPIRLGLALNFSVFYYEILNNPELACTLAKTAFDEAIAELDTLNEDSYKDSTLIMQLLRDNLTLWTSDSAGEECDAAEGAEN

SEQ ID NO: 5 - protein, 154 aa, Homo sapiens
YWHAQMEKTELIQKAKLAEQAERYDDMATCMKAVTEQGAELSNEERNLLSVAYKNVVGGRRSAWRVISSIEQKTDTSDKKLQLIKDYREKVESELRSICTTVLELLDKYLIANATNPESKVFYLKMKGDYFRYLAEVACGDDRKQTIDNSQGAY

SEQ ID NO: 6 - protein, 295 aa, Homo sapiens
MSGALDVLQMKEEDVLKFLAAGTHLGGTNLDFQMEQYIYKRKSDGIYIINLKRTWEKLLLTARAIVAIENPADVSVISSRNTGQRAVLKFAAATGATPIAGRFTPGTFTNQIQAAFREPRLLVVSDPRADHQPLTEASYVNLPTIALCNTDSPLHYVDIAIPCNNKGTHSVGLMWWMLAREVLRMRGTISREHPWEVMPDLYFYRDPEEIEKEEQAAAEKAMTREELQGEWTAPAPEFTATQPEVADWSEGVQVPSVPIQQFPTEDWSTQRATEDWSAAPTAQATEWVGATTDWS

SEQ ID NO: 7 - protein, 300 aa, Homo sapiens
MSGALDVLQMKEEDVLKFLAAGTHLGGTNLDFQMEQYIYKRKSDGIYIINLKRTWEKLLLAARAIVAIENPADVSVISSRNTGQVCGTVRAVLKFAAATGATPIAGRFTPGTFTNQIQAAFREPRLLVVTDPRADHQPLTEASYVNLPTIALCNTDSPLRYVDIAIPCNNKGAHSVGLMWWMLAREVLRMRGTISREHPWEVMPDLYFYRDPEEIEKEEQAAAEKAVTKEEFQGEWTAPAPEFTATQPEVADWSEGVQVPSVPIQQFPTEDWSAQPATEDWSAAPTAQATEWVGATTDWS

SEQ ID NO: 8 - protein, 295 aa, Homo sapiens [sequence omitted]

SEQ ID NO: 9 - protein, 147 aa, Homo sapiens
MSGALDVLQMKEEDVLKFLAAGTHLGGTNLDFQMEQYIYKRKSDGIYIINLKRTWEKLLLAARAIVAIENPADVSVISSRNTGQRAVLKFAAATGATPIAGRFTPGTFTNQIQAAFREPRLLVVTDPRADHQPLTEASYVNLPTIAL

SEQ ID NO: 10 - protein, 213 aa, Homo sapiens
MSGALDVLQMKEEDVLKFLAAGTHLGGTNLDFQMEQYIYKRKSDGIYIINLKRTWEKLLLAARAIVAIENPADVSVISSRNTGQGAHSVGLMWWMLAREVLRMRGTISREHPWEVMPDLYFYRDPEEIEKEEQAAAEKAVTKEEFQGEWTAPAPEFTATQPEVADWSEGVQVPSVPIQQFPTEDWSAQPATEDWSAAPTAQATEWVGATTDWS

SEQ ID NO: 11 - protein, 316 aa, Homo sapiens
MATFVELSTKAKMPIVGLGTWKSPLGKVKEAVKVAIDAGYRHIDCAYVYQNEHEVGEAIQEKIQEKAVKREDLFIVSKLWPTFFERPLVRKAFEKTLKDLKLSYLDVYLIHWPQGFKSGDDLFPKDDKGNAIGGKATFLDAWEAMEELVDEGLVKALGVSNFSHFQIEKLLNKPGLKYKPVTNQVECHPYLTQEKLIQYCHSKGITVTAYSPLGSPDRPWAKPEDPSLLEDPKIKEIAAKHKKTAAQVLIRFHIQRNVIVIPKSVTPARIVENIQVFDFKLSDEEMATILSFNRNWRACNVLQSSHLEDYPFDAEY

SEQ ID NO: 12 - protein, 430 aa, Homo sapiens
MALLHSGRVLPGIAAAFHPGLAAAASARASSWWTHVEMGPPDPILGVTEAFKRDTNSKKMNLGVGAYRDDNGKPYVLPSVRKAEAQIAAKNLDKEYLPIGGLAEFCKASAELALGENSEVLKSGRFVTVQTISGTGALRIGASFLQRFFKFSRDVFLPKPTWGNHTPIFRDAGMQLQGYRYYDPKTCGFDFTGAVEDISKIPEQSVLLLHACAHNPTGVDPRPEQWKEIATVVKKRNLFAFFDMAYQGFASGDGDKDAWAVRHFIEQGINVCLCQSYAKNMGLYGERVGAFTMVCKDADEAKRVESQLKILIRPMYSNPPLNGARIAAAILNTPDLRKQWLQEVKGMADRIIGMRTQLVSNLKKEGSTHNWQHITDQIGMFCFTGLKPEQVERLIKEFSIYMTKDGRISVAGVTSSNVGYLAHAIHQVTK

SEQ ID NO: 13 - protein, 633 aa, Homo sapiens
MANQVNGNAVQLKEEEEPMDTSSVTHTEHYKTLIEAGLPQKVAERLDEIFQTGLVAYVDLDERAIDALREFNEEGALSVLQQFKESDLSHVQNKSAFLCGVMKTYRQREKQGSKVQESTKGPDEAKIKALLERTGYTLDVTTGQRKYGGPPPDSVYSGVQPGIGTEVFVGKIPRDLYEDELVPLFEKAGPIWDLRLMMDPLSGQNRGYAFITFCGKEAAQEAVKLCDSYEIRPGKHLGVCISVANNRLFVGSIPKNKTKENILEEFSKVTEGLVDVILYHQPDDKKKNRGFCFLEYEDHKSAAQARRRLMSGKVKVWGNVVTVEWADPVEEPDPEVMAKVKVLFVRNLATTVTEEILEKSFSEFGKLERVKKLKDYAFVHFEDRGAAVKAMDEMNGKEIEGEEIEIVLAKPPDKKRKERQAARQASRSTAYEDYYYHPPPRMPPPIRGRGRGGGRGGYGYPPDYYGYEDYYDDYYGYDYHDYRGGYEDPYYGYDDGYAVRGRGGGRGGRGAPPPPRGRGAPPPRGRAGYSQRGAPLGPPRGSRGGRGGPAQQQRGRGSRGSRGNRGGNVGGKRKADGYNQPDSKRRQTNNQQNWGSQPIAQQPLQQGGDYSGNYGYNNDNQEFYQDTYGQQWK

SEQ ID NO: 14 - protein, 505 aa, Homo sapiens
MRLRRLALFPGVALLLAAARLAAASDVLELTDDNFESRISDTGSAGLMLVEFFAPWCGHCKRLAPEYEAAATRLKGIVPLAKVDCTANTNTCNKYGVSGYPTLKIFRDGEEAGAYDGPRTADGIVSHLKKQAGPASVPLRTEEEFKKFISDKDASIVGFFDDSFSEAHSEFLKAASNLRDNYRFAHTNVESLVNEYDDNGEGIILFRPSHLTNKFEDKTVAYTEQKMTSGKIKKFIQENIFGICPHMTEDNKDLIQGKDLLIAYYDVDYEKNAKGSNYWRNRVMMVAKKFLDAGHKLNFAVASRKTFSHELSDFGLESTAGEIPVVAIRTAKGEKFVMQEEFSRDGKALERFLQDYFDGNLKRYLKSEPIPESNDGPVKVVVAENFDEIVNNENKDVLIEFYAPWCGHCKNLEPKYKELGEKLSKDPNIVIAKMDATANDVPSPYEVRGFPTIYFSPANKKLNPKKYEGGRELSDFISYLQREATNPPVIQEEKPKKKKKAQEDL

SEQ ID NO: 15 - protein, 152 aa, Homo sapiens
MANLERTFIAIKPDGVQRGLVGEIIKRFEQKGFRLVAMKFLRASEEHLKQHYIDLKDRPFFPGLVKYMNSGPVVAMVWEGLNVVKTGRVMLGETNPADSKPGTIRGDFCIQVGRNIIHGSDSVKSAEKEISLWFKPEELVDYKSCAHDWVYE

SEQ ID NO: 16 - protein, 1192 aa, Homo sapiens
MEDLDQSPLVSSSDSPPRPQPAFKYQFVREPEDEEEEEEEEEEDEDEDLEELEVLERKPAAGLSAAPVPTAPAAGAPLMDFGNDFVPPAPRGPLPAAPPVAPERQPSWDPSPVSSTVPAPSPLSAAAVSPSKLPEDDEPPARPPPPPPASVSPQAEPVWTPPAPAPAAPPSTPAAPKRRGSSGSVDETLFALPAASEPVIRSSAENMDLKEQPGNTISAGQEDFPSVLLETAASLPSLSPLSAASFKEHEYLGNLSTVLPTEGTLQENVSEASKEVSEKAKTLLIDRDLTEFSELEYSEMGSSFSVSPKAESAVIVANPREEIIVKNKDEEEKLVSNNILHNQQELPTALTKLVKEDEVVSSEKAKDSFNEKRVAVEAPMREEYADFKPFERVWEVKDSKEDSDMLAAGGKIESNLESKVDKKCFADSLEQTNHEKDSESSNDDTSFPSTPEGIKDRSGAYITCAPFNPAATESIATNIFPLLGDPTSENKTDEKKIEEKKAQIVTEKNTSTKTSNPFLVAAQDSETDYVTTDNLTKVTEEVVANMPEGLTPDLVQEACESELNEVTGTKIAYETKMDLVQTSEVMQESLYPAAQLCPSFEESEATPSPVLPDIVMEAPLNSAVPSAGASVIQPSSSPLEASSVNYESIKHEPENPPPYEEAMSVSLKKVSGIKEEIKEPENINAALQETEAPYISIACDLIKETKLSAEPAPDFSDYSEMAKVEQPVPDHSELVEDSSPDSEPVDLFSDDSIPDVPQKQDETVMLVKESLTETSFESMIEYENKEKLSALPPEGGKPYLESFKLSLDNTKDTLLPDEVSTLSKKEKIPLQMEELSTAVYSNDDLFISKEAQIRETETFSDSSPIEIIDEFPTLISSKTDSFSKLAREYTDLEVSHKSEIANAPDGAGSLPCTELPHDLSLKNIQPKVEEKISFSDDFSKNGSATSKVLLLPPDVSALATQAEIESIVKPKVLVKEAEKKLPSDTEKEDRSPSAIFSAELSKTSVVDLLYWRDIKKTGVVFGASLFLLLSLTVFSIVSVTAYIALALLSVTISFRIYKGVIQAIQKSDEGHPFRAYLESEVAISEELVQKYSNSALGHVNCTIKELRRLFLVDDLVDSLKFAVLMWVFTYVGALFNGLTLLILALISLFSVPVIYERHQAQIDHYLGLANKNVKDAMAKIQAKIPGLKRKAE

SEQ ID NO: 17 - protein, 213 aa, Homo sapiens
MSVELEEALPVTTAEGMAKKVTKAGGSAALSPSKKRKNSKKKNQPGKYSQLVVETIRRLGERNGSSLAKIYTEAKKVPWFDQQNGRTYLKYSIKALVQNDTLLQVKGTGANGSFKLNRKKLEGGGERRGAPAAATAPAPTAHKAKKAAPGAAGSRRADKKPARGQKPEQRSHKKGAGAKKDKGGKAKKTAAAGGKKVKKAAKPSVPKVPKGRK

SEQ ID NO: 18 - protein, 466 aa, Homo sapiens
MVMEKPSPLLVGREFVRQYYTLLNQAPDMLHRFYGKNSSYVHGGLDSNGKPADAVYGQKEIHRKVMSQNFTNCHTKIRHVDAHATLNDGVVVQVMGLLSNNNQALRRFMQTFVLAPEGSVANKFYVHNDIFRYQDEVFGGFVTEPQEESEEEVEEPEERQQTPEVVPDDSGTFYDQAVVSNDMEEHLEEPVAEPEPDPEPEPEQEPVSEIQEEKPEPVLEETAPEDAQKSSSPAPADIAQTVQEDLRTFSWASVTSKNLPPSGAVPVTGIPPHVVKVPASQPRPESKPESQIPPQRPQRDQRVREQRINIPPQRGPRPIREAGEQGDIEPRRMVRHPDSHQLFIGNLPHEVDKSELKDFFQSYGNVVELRINSGGKLPNFGFVVFDDSEPVQKVLSNRPIMFRGEVRLNVEEKKTRAAREGDRRDNRLRGPGGPRGGLGGGMRGPPRGGMVQKPGFGVGRGLAPRQ

SEQ ID NO: 19 - protein, 732 aa, Homo sapiens
MPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 20 - protein, 911 aa, Homo sapiens
MVDYHAANQSYQYGPSSAGNGAGGGGSMGDYMAQEDDWDRDLLLDPAWEKQQRKTFTAWCNSHLRKAGTQIENIDEDFRDGLKLMLLLEVISGERLPKPERGKMRVHKINNVNKALDFIASKGVKLVSIGAEEIVDGNAKMTLGMIWTIILRFAIQDISVEETSAKEGLLLWCQRKTAPYKNVNVQNFHISWKDGLAFNALIHRHRPELIEYDKLRKDDPVTNLNNAFEVAEKYLDIPKMLDAEDIVNTARPDEKAIMTYVSSFYHAFSGAQKAETAANRICKVLAVNQENEHLMEDYEKLASDLLEWIRRTIPWLEDRVPQKTIQEMQQKLEDFRDYRRVHKPPKVQEKCQLEINFNTLQTKLRLSNRPAFMPSEGKMVSDINNGWQHLEQAEKGYEEWLLNEIRRLERLDHLAEKFRQKASIHEAWTDGKEAMLKHRDYETATLSDIKALIRKHEAFESDLAAHQDRVEQIAAIAQELNELDYYDSHNVNTRCQKICDQWDALGSLTHSRREALEKTEKQLEAIDQLHLEYAKRAAPFNNWMESAMEDLQDMFIVHTIEEIEGLISAHDQFKSTLPDADREREAILAIHKEAQRIAESNHIKLSGSNPYTTVTPQIINSKWEKVQQLVPKRDHALLEEQSKQQSNEHLRRQFASQANVVGPWIQTKMEEIGRISIEMNGTLEDQLSHLKQYERSIVDYKPNLDLLEQQHQLIQEALIFDNKHTNYTMEHIRVGWEQLLTTIARTINEVENQILTRDAKGISQEQMQEFRASFNHFDKDHGGALGPEEFKACLISLGYDVENDRQGEAEFNRIMSLVDPNHSGLVTFQAFIDFMSRETTDTDTADQVIASFKVLAGDKNFITAEELRRELPPDQAEYCIARMAPYQGPDAVPGALDYKSFSTALYGESDL

(19) United States
(12) Patent Application Publication
(10) Pub. No.: US 2012/0100558 A1
(43) Pub. Date: Apr. 26, 2012

(54) LUNG CANCER DIAGNOSIS

(76) Inventors: Samir M. Hanash, Mercer Island, WA (US); Ji Qiu, Chandler, AZ (US); Sandra Faca, Sao Paolo (BR)

(21) Appl. No.: 13/062,929

(22) PCT Filed: Aug. 27, 2009
(86) PCT No.: PCT/US2009/055190

§ 371 Date: Aug. 4, 2011

Related U.S. Application Data
(60) Provisional application No. 61/095,269, filed on Sep. 8, 2008.

(51) Int. Cl. G01N 33/53

(52) U.S. Cl. 435/7.9; 436/501

(57) ABSTRACT

Diagnosis of lung cancer in a subject before onset of symptoms is described herein (i.e., in a pre-diagnostic subject), by screening a biological fluid from the subject for the presence therein of autoantibodies that are specific for one or more pre-diagnostic lung cancer indicator proteins, including LAMR1, and optionally additionally or alternatively including annexin I and/or 14-3-3-theta and/or other pre-diagnostic lung cancer indicator proteins as presently disclosed, as the defined antigens. Related methods, including for monitoring immune reactivity against lung cancer indicator proteins in a lung cancer patient, typing lung cancer subjects or characterizing lung tumors, and application of the described proteomics approach for the identification of additional pre-diagnostic lung cancer indicator proteins, are also contemplated.

### CROSS REFERENCE TO RELATED APPLICATION

This application claims the benefit of U.S. Provisional Application Ser. No. 61/095,269, filed Sep. 8, 2008.

### STATEMENT OF GOVERNMENT INTEREST

This invention was made with government support under Grant No. UO1 CA084982, awarded by the National Cancer Institute/National Institutes of Health. The government has certain rights in this invention.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360056_{—}404PC_SEQUENCE_LISTING.txt. The text file is 65 KB, was created on Aug. 27, 2009 and is being submitted electronically via EFS-Web to the U.S. PCT Receiving Office, concurrent with the filing of the specification.

### BACKGROUND

1. Technical Field

The presently disclosed invention embodiments relate to compositions and methods for the early detection of cancer. In particular, certain embodiments relate to compositions and methods for diagnosing lung cancer in patients otherwise exhibiting no signs of cancer, by detecting in patient samples the presence of autoantibodies that are specific for one or more lung cancer indicator proteins as described herein.

2. Description of the Related Art

Lung cancer accounts for over 12% of all human cancers and close to 18% of cancer deaths, thus representing one of the most commonly occurring cancers in humans worldwide. (World Cancer Research Fund/American Institute for Cancer Research. Food, Nutrition and the Prevention of Cancer: A global perspective. Washington, D.C.: American Institute for Cancer Research, 2007.) Non-small cell lung cancer is most prevalent, including squamous cell carcinoma (˜30-45% of all lung cancers), adenocarcinoma (˜30-45%) and large cell carcinoma (˜9%). Small-cell carcinoma represents approximately 10-15% of all lung cancers. In 2002, approximately 1.4 million lung cancer cases were reported. Id.

In its early stages, lung cancer produces no symptoms, thereby typically eluding detection until the disease has progressed to advanced stages that are associated with high mortality rates.

Currently available non-invasive approaches for screening and diagnosis of lung cancer include chest x-rays and other imaging methods such as computerized tomography (CT), for example, low-dose helical computed tomography. These approaches have not, however, been shown to reduce lung cancer mortality rates, and may generate false positive results that could lead to unnecessary and potentially harmful invasive procedures and/or therapeutic regimens. (Lung Cancer Screening PDQ® Summary, National Cancer Institute, National Institutes of Health, Bethesda, Md., 2008) Additionally or alternatively, cytological analysis of sputum samples may be performed in efforts to detect cancer cells, typically for patients presenting with productive cough, a symptom which may signify the presence of an advanced case of lung cancer.

Invasive procedures to obtain lung biopsy samples may also be performed in efforts to diagnose lung cancer, including procedures such as bronchoscopy, mediastinoscopy or imaging-guided needle biopsy. These procedures are typically practiced only in patients presenting with one or more potential signs or symptoms of advanced lung cancer (e.g., a mass or nodule appearing in x-ray of CT imaging studies), which may warrant the time and expense of such procedures and of the subsequent diagnostic work-up of the biopsy samples. Hence, these procedures are not amenable to routine screening for, or early detection of, lung cancer.

There is increasing evidence for a humoral immune response to cancer in humans, as demonstrated by the identification of autoantibodies against a number of intracellular and surface antigens in patients with various tumor types (Stocked et al., J Exp Med 187:1349-54, 1998; Tan E M, J Clin Invest 108:1411-5, 2001; Mintz et al., Nat Biotechnol 21:57-63, 2003; Gourevitch et al., Br J Cancer 72:934-8, 1995; Gure et al., Cancer Res 58:1034-41, 1998; Yamamoto et al., Int J Cancer 69:283-9, 1996; Dunn et al., Nat Immunol 3:991-8, 2002; Hanash, Nat Biotechnol 21:37-8, 2003; Old et al., J Exp Med 187:1163-7, 1998; Finn, N Engl J Med 353:1288-90, 2005). Interest in the humoral response against tumor antigens relates in part to the potential screening and diagnostic utility of autoantibodies and their corresponding antigens.

A number of circulating autoantibodies in lung cancer have been identified by screening expression libraries with patient sera (Gure et al., Cancer Res 58:1034-41, 1998; Yamamoto et al., Int J Cancer 69:283-9, 1996; Diesinger et al., Int J Cancer 102:372-8, 2002; Gure et al., Proc Natl Acad Sci USA 97:4198-203, 2000; Ali Eldib et al., Int J Cancer 108:558-63, 2004; Yang et al., J Proteome Res 6:751-8, 2007). However, there remains a need to identify additional autoantibody targets to increase specificity and sensitivity.

Several proteomics methods are emerging as useful means for discovering autoantibody biomarkers (e.g., Hanash, Nature 422:226-32, 2003; Imafuku, Omenn and Hanash, Dis Markers 20:149-53, 2004; U.S. Pat. Nos. 6,645,465; 7,202,045; 7,387,881). The merit of a proteomic approach is that it allows identification of autoantibodies to proteins that are directly derived from cancer cells or tumors and thus may uncover antigenicity associated with proteins as they occur in tumor cells, including proteins whose antigenicities have structural bases in their post-translational modification. Previous studies using two-dimensional gels of lung tumor cell lysates and Western blotting uncovered autoantibodies in lung cancer patient sera against annexin I, PGP9.5 and 14-3-3 theta proteins (Brichory et al., Cancer Res 61:7908-12, 2001; Brichory et al., Proc Natl Acad Sci USA 98:9824-9, 2001; Pereira-Faca et al., Cancer Res 67:12000-6, 2007).

More recently, a method has been implemented that utilized liquid-based procedures to separate intact proteins in tissue and tumor cell lysates (Wang and Hanash, J Chromatogr B Analyt Technol Biomed Life Sci 787:11-8, 2003; Faca et al., J Proteome Res 6:3558-65, 2007). Several hundreds of distinct protein-containing fractions were spotted onto microarrays, interrogated using various sources of sera, and quantitatively analyzed for bound antibodies. Anti-PGP9.5 antibodies were successfully identified in sera of newly diagnosed lung cancer patients, and anti-UCHL3 antibodies were identified in colon cancer patient sera collected at the time of diagnosis, using this microarray approach (Nam et al., Proteomics 3:2108-15, 2003; Madoz-Gurpide et al., Mol Cell Proteomics, 2007), as well as autoantibodies in prostate cancer (Forrester et al., Proteomics—Clinical Applications 1:494-505, 2007), thus establishing the potential of natural protein microarrays to uncover antigens that induce an antibody response in cancer in a relatively high throughput approach.

Clearly there remains a significant unmet need for more and better compositions and methods for diagnosing lung cancer and for lung cancer screening, including additional biomarkers and conveniently practiced approaches that are capable of detecting lung cancer in a subject at an earlier point in the progression of the disease than is currently possible (e.g., in a pre-diagnostic subject), and preferably further including conveniently practiced methods that permit monitoring the severity of disease, progression of disease and/or disease responsiveness to a therapeutic course. Effective early diagnosis of lung cancer would reduce the tumor burden that is typically present at the inception of surgical, chemotherapeutic, immunotherapeutic, molecular therapeutic and/or radiation-based therapies, relative to the tumor burden presently confronting the clinician when current conventional diagnostics are relied upon, and could have a significant impact on lung cancer related mortality. The present invention thus addresses these needs and offers other related advantages.

### BRIEF SUMMARY

In one embodiment, the present invention provides a method for diagnosing lung cancer in a pre-diagnostic subject, comprising (a) contacting (i) one or more antibodies from a biological fluid from the pre-diagnostic subject, and (ii) at least one isolated pre-diagnostic lung cancer indicator protein, under conditions and for a time sufficient for detecting specific binding of at least one antibody from the biological fluid to one or more of said pre-diagnostic lung cancer indicator proteins, and therefrom identifying presence of lung cancer in the pre-diagnostic subject.

In another embodiment, there is provided a screening method for lung cancer, comprising (a) contacting (i) one or more antibodies from a biological fluid from each subject of one or a plurality of subjects, and (ii) at least one isolated pre-diagnostic lung cancer indicator protein, under conditions and for a time sufficient for detecting specific binding of at least one antibody from the biological fluid to one or more of said pre-diagnostic lung cancer indicator proteins, wherein detection of specific binding indicates the subject has lung cancer, and thereby screening for lung cancer.

In another embodiment there is provided a method for diagnosing lung cancer in a pre-diagnostic subject, comprising (a) contacting (i) one or more antibodies from a biological fluid from the pre-diagnostic subject, and (ii) an isolated protein or polypeptide that comprises one or more antigenic epitopes of one or more pre-diagnostic lung cancer indicator proteins, under conditions and for a time sufficient for detecting specific binding of at least one antibody from the biological fluid to one or more of said antigenic epitopes, and therefrom identifying presence of lung cancer in the pre-diagnostic subject.

In another embodiment there is provided a screening method for lung cancer, comprising (a) contacting (i) one or more antibodies from a biological fluid from each subject of one or a plurality of subjects, and (ii) an isolated protein or polypeptide that comprises one or more antigenic epitopes of one or more pre-diagnostic lung cancer indicator proteins, under conditions and for a time sufficient for detecting specific binding of at least one antibody from the biological fluid to one or more of said antigenic epitopes, wherein detection of specific binding indicates the subject has lung cancer, and thereby screening for lung cancer.

In certain further embodiments of the above described methods, at least one of the one or more pre-diagnostic lung cancer indicator proteins comprises a LAMR1 protein, AKR1B10 protein [SEQ ID NO:11], GOT2 protein [SEQ ID NO:12], HNRPR protein [SEQ ID NO:13], PDIA3 protein [SEQ ID NO:14], NME2 protein [SEQ ID NO:15], RTN4 protein [SEQ ID NO:16], HI1FX protein [SEQ ID NO:17], G3BP protein [SEQ ID NO:18], HSPCA protein [SEQ ID NO:19], or ACTN4 protein [SEQ ID NO:20]. In certain still further embodiments the pre-diagnostic lung cancer indicator proteins further comprise at least one, two, three, four, five, six, seven, eight or more proteins selected from annexin I protein, 14-3-3 theta protein, AKR1B10 protein [SEQ ID NO:11], GOT2 protein [SEQ ID NO:12], HNRPR protein [SEQ ID NO:13], PDIA3 protein [SEQ ID NO:14], NME2 protein [SEQ ID NO:15], RTN4 protein [SEQ ID NO:16], HI1FX protein [SEQ ID NO:17], G3BP protein [SEQ ID NO:18], HSPCA protein [SEQ ID NO:19], and ACTN4 protein [SEQ ID NO:20].

In certain other further embodiments the lung cancer is selected from (i) adenocarcinoma, (ii) squamous cell carcinoma, (iii) non-small cell lung cancer that is not (i) or (ii), and (iv) a lung cancer that can be defined based on one or more of causation and gene mutational status. In certain other further embodiments the subject or pre-diagnostic subject is at increased risk for developing lung cancer. In certain further embodiments the subject or pre-diagnostic subject has at least one indicator of increased risk for developing lung cancer that is selected from (i) a history of asbestos exposure, (ii) a history of smoking tobacco products, (iii) a history of radon gas exposure, (iv) a history of exposure to a source of ionizing radiation, (v) a history of recurrent lung inflammation, (vi) a history of tuberculosis, (vi) a history of silicosis, berylliosis or talc inhalation, (vii) a family history of lung cancer in genetically related individuals, (viii) a history of vitamin A deficiency or vitamin A excess, (ix) a history of smoking cannabis, and (x) exposure to toxic volatile substances or infectious agents.

In certain embodiments of the above described methods, the antibodies are isolated from the biological fluid prior to the step of contacting, and in certain other embodiments the antibodies are present in the biological fluid during the step of contacting. In certain embodiments the antibodies are autoantibodies. In certain embodiments of the above described methods, the biological fluid is selected from blood, serum, serosal fluid, plasma, lymph, urine, cerebrospinal fluid, saliva, a mucosal secretion, a vaginal secretion, ascites fluid, pleural fluid, pericardial fluid, peritoneal fluid, abdominal fluid, culture medium, conditioned culture medium and lavage fluid. In certain embodiments the biological fluid comprises serum.

In certain other embodiments of the above described methods, the pre-diagnostic indicator protein, or the isolated protein or polypeptide that comprises one or more antigenic epitopes of a pre-diagnostic indicator protein, is selected from (i) a naturally occurring protein or polypeptide, (ii) a synthetic protein or polypeptide, (iii) a recombinant protein or polypeptide, and (iv) a fusion protein or polypeptide that comprises a fusion polypeptide domain fused to the pre-diagnostic indicator protein, or to the polypeptide that comprises one or more antigenic epitopes of the pre-diagnostic indicator protein.

In certain other embodiments of the above described methods, the pre-diagnostic indicator protein, or the isolated protein or polypeptide that comprises one or more antigenic epitopes of a pre-diagnostic indicator protein, is immobilized on a solid substrate. In certain further embodiments the immobilized pre-diagnostic indicator protein or the immobilized isolated protein or polypeptide that comprises one or more antigenic epitopes of a pre-diagnostic indicator protein, is immobilized by a covalent bond. In certain other further embodiments the immobilized pre-diagnostic indicator protein or the immobilized isolated protein or polypeptide that comprises one or more antigenic epitopes of a pre-diagnostic indicator protein, is non-covalently immobilized.

In certain other embodiments of the above described methods, detecting specific binding of the at least one antibody comprises detecting a signal that is selected from a fluorescent signal, a radiometric signal, an enzymatic signal and a spectrometric signal.

In certain other embodiments of the above described methods, the pre-diagnostic lung cancer indicator protein is selected from the group consisting of (i) a non-posttranslationally modified protein, (ii) a posttranslationally modified protein that is selected from a glycoprotein, a lipoprotein, a phosphoprotein, a proteolipid, a glypiated protein, a ubiquitinylated protein, a SUMOylated protein, a sulfated protein and a glycated protein, and (iii) a posttranslationally modified protein of (ii) in which one or more posttranslational modifications results in immunogenicity.

In another embodiment there is provided a method of monitoring lung cancer autoimmune reactivity in a lung cancer patient, comprising (a) contacting, after each of two or more timepoints, (i) one or more antibodies from a biological fluid that is taken from a subject at each of said timepoints, and (ii) a test antigen that is selected from the group consisting of (1) at least one isolated pre-diagnostic lung cancer indicator protein and (2) at least one isolated protein or polypeptide that comprises one or more antigenic epitopes of one or more pre-diagnostic lung cancer indicator proteins, under conditions and for a time sufficient for detecting specific binding of at least one antibody from the biological fluid to one or more of said pre-diagnostic lung cancer indicator proteins or antigenic epitopes thereof; and (b) comparing the specific binding that is detectable by antibodies from the biological fluid taken at each of said two or more timepoints, and thereby monitoring lung cancer autoimmune reactivity in the patient. In certain further embodiments, a first timepoint occurs before administration of a therapeutic agent to the patient and a second timepoint occurs after administration of the therapeutic agent to the patient.

These and other aspects of the invention will be evident upon reference to the following detailed description and attached drawings. All of the U.S. patents, U.S. patent application publications, U.S. patent applications, foreign patents, foreign patent applications and non-patent publications referred to in this specification and/or listed in the Application Data Sheet, are incorporated herein by reference in their entirety, as if each was incorporated individually. Aspects of the invention can be modified, if necessary, to employ concepts of the various patents, applications and publications to provide yet further embodiments of the invention.

### BRIEF DESCRIPTION OF THE DRAWINGS

FIG. 1 shows reproducibility of natural protein microarrays. (FIG. 1A), Quantitative reproducibility was assessed by hybridization of the same pooled sample on 6 different microarrays. (FIG. 1B), Pearson correlations between replicate spots on each of the 6 different microarrays were all 0.99 and correlations between replicate microarrays were greater than 0.96. Representative scatter plots were presented.

FIG. 2 shows reactivity of annexin I (FIG. 2A), 14-3-3 theta (FIG. 2B), PGP9.5 (FIG. 2C) and LAMR1 (FIG. 2D) containing spotted fractions with all 85+85 CARET sera.

FIG. 3 shows combined ROC analysis of the LAMR1, annexin 1 and 14-3-3 theta fractions based on reactivity with all 85+85 CARET sera. The hatched 95% confidence band in the plot was estimated from 500 bootstraps.

### DETAILED DESCRIPTION

The presently disclosed invention embodiments derive from the surprising discovery, in pre-diagnostic subjects, of autoantibodies that specifically react with one or more pre-diagnostic lung cancer indicator proteins as described herein. These embodiments offer unprecedented advantages associated with early detection of lung cancer in a subject at a time when the subject otherwise exhibits none of the previously recognized signs or symptoms of lung cancer. Accordingly, these and related embodiments will find uses in screening methods for lung cancer and in methods for diagnosing lung cancer in pre-diagnostic subjects, and in other related methods.

In particular, and as described in greater detail below, it has been found unexpectedly that in a biological fluid from a pre-diagnostic subject, for instance, a subject in whom lung cancer cannot be diagnosed by any other means, detection (i) of autoantibodies specific for a LAMR1 protein, (ii) of autoantibodies specific for a LAMR1 protein and autoantibodies specific for an annexin I protein, (iii) of autoantibodies specific for a LAMR1 protein and autoantibodies specific for a 14-3-3 theta protein, (iv) of autoantibodies specific for a LAMR1 protein and autoantibodies specific for an annexin I protein and autoantibodies specific for a 14-3-3 theta protein, or (v) of autoantibodies specific for at least one, two, three, four, five, six, seven, eight or more proteins selected from a LAMR1 protein, annexin I protein, 14-3-3 theta protein, AKR1B10 protein [SEQ ID NO:11], GOT2 protein [SEQ ID NO:12], HNRPR protein [SEQ ID NO:13], PDIA3 protein [SEQ ID NO:14], NME2 protein [SEQ ID NO:15], RTN4 protein [SEQ ID NO:16], HI1FX protein [SEQ ID NO:17], G3BP protein [SEQ ID NO:18], HSPCA protein [SEQ ID NO:19], and ACTN4 protein [SEQ ID NO:20], indicates the presence of lung cancer in the subject. Detection of such autoantibodies in pre-diagnostic subjects precedes the subsequent onset of lung cancer symptoms and/or the subsequent ability to diagnose lung cancer by other diagnostic means.

Without wishing to be bound by theory, it is believed according to the present disclosure that at a very early stage of lung cancer, and in particular in lung cancer that cannot be detected in a subject by any previously available means for detecting lung cancer (e.g., imaging or biopsy, or by the appearance of clinical signs or symptoms), expression of one or more pre-diagnostic lung cancer indicator proteins, by lung cancer cells and/or by precancerous cells that are inexorably committed to progression into lung cancer cells and/or by other cells associated with such lung cancer or precancerous cells, elicits an immune response in the subject that results in the production of one or more detectable antibodies that specifically bind to the one or more pre-diagnostic lung cancer indicator proteins. Further according to non-limiting theory, these antibodies that specifically bind to one or more pre-diagnostic lung cancer indicator proteins are autoantibodies that are present in a biological fluid in the pre-diagnostic subject.

Accordingly, certain of the preferred embodiments disclosed herein contemplate a simple, non-invasive diagnostic assay whereby a biological fluid from a pre-diagnostic subject may be tested for the presence of antibodies (e.g., autoantibodies) that specifically bind to one or more pre-diagnostic lung cancer indicator proteins as provided herein, and/or that specifically bind to a polypeptide that comprises one or more antigenic epitopes of one or more pre-diagnostic lung cancer indicator proteins as provided herein. These and related embodiments provide screening and diagnostic methods for lung cancer that are inexpensive, readily amenable to screening a plurality of subjects such as in a high-throughput format, and that offer the advantage of lung cancer detection at an earlier stage in the onset and progression of disease than is afforded by any previously existing technology.

The methods described herein may therefore be used to diagnose lung cancer in a pre-diagnostic subject and/or to screen one or a plurality of subjects for lung cancer, which may include any cancer, tumor, neoplasia, malignancy or other cancer present in the lung, whether by virtue of having originated in the lung as a spontaneous or primary tumor, or by having metastasized, invaded, lodged or otherwise migrated to the lung from a different site. The lung cancer that is detected according to the presently disclosed methods may therefore be a non-small cell lung cancer such as squamous cell carcinoma, adenocarcinoma, large cell carcinoma or other non-small cell carcinoma, and may also be a small-cell carcinoma, but the invention is not intended to be so limited and also contemplates other lung cancers. For example, regardless of whether or not a particular lung cancer can be identified as being a non-small cell lung cancer or small-cell carcinoma, certain embodiments also contemplate a lung cancer that can additionally or alternatively be defined (e.g., typed, classified, characterized or otherwise identified according to art-accepted criteria) based on its causation and/or its gene mutational status. Thus, and as known in the art and discussed in certain of the publications cited herein, causal or causative factors such as exposure to environmental insults including but not limited to toxins, carcinogens, radiation, free radicals, infectious agents and/or other agents of cancer causation, and/or inherent or acquired genetic mutations at any one or more of a large number of known genetic loci in which particular mutations have been linked to cancer (e.g., p53), may underlie certain lung cancers that may but need not also be amenable to classification by other criteria.

Certain preferred embodiments contemplate a subject or biological source that is a human subject such as a pre-diagnostic subject, which includes a subject in whom lung cancer is not detectable by one or more art-accepted diagnostic methods for lung cancer that were in use prior the present disclosure. In certain embodiments, the herein described methods may be practiced using a biological fluid as provided herein from a patient that has been classified as being at risk for developing or acquiring lung cancer according to art-accepted clinical diagnostic criteria, and in certain embodiments the patient has been diagnosed as having lung cancer using previously described diagnostic criteria, which include criteria by which a given lung cancer may be typed as, e.g., small-cell or large-cell lung cancer, or as squamous cell carcinoma, adenocarcinoma, etc., such as the criteria of the U.S. National Cancer Institute (Bethesda, Md., USA) or as described in DeVita, Hellman, and Rosenberg's Cancer: Principles and Practice of Oncology (2008, Lippincott, Williams and Wilkins, Philadelphia/Ovid, New York); Pizzo and Poplack, Principles and Practice of Pediatric Oncology (Fourth edition, 2001, Lippincott, Williams and Wilkins, Philadelphia/Ovid, New York); and Vogelstein and Kinzler, The Genetic Basis of Human Cancer (Second edition, 2002, McGraw Hill Professional, New York). Certain embodiments contemplate a human subject that is known to be free of a risk for having, developing or acquiring cancer by such criteria.

In certain preferred embodiments of the invention, the subject or biological source may be suspected of having or being at risk for having a malignant condition, and in certain preferred embodiments of the invention the subject or biological source may be known to be free of a risk or presence of such disease. Certain embodiments contemplate performing the methods described herein using a biological fluid from a pre-diagnostic subject, e.g., a subject in whom lung cancer is not detectable by one or more art-accepted diagnostic methods for lung cancer that were in use prior the present disclosure.

Those familiar with the art will therefore appreciate that a subject, including a pre-diagnostic subject, from whom a biological fluid may be obtained in order to practice certain herein described screening and/or diagnostic methods, may be at increased (i.e., in a statistically significant manner relative to appropriate controls) risk for developing lung cancer, for example, even where no frank signs or symptoms of lung cancer are apparent. A subject having an increased risk for developing lung cancer typically exhibits one or more indicators of increased risk for developing lung cancer. These indicators of increased risk are known in the art and can be readily determined, and include but need not be limited to (i) a history of asbestos exposure, (ii) a history of smoking tobacco products including exposure to second-hand smoke, (iii) a history of radon gas exposure, (iv) a history of exposure to a source of ionizing radiation, (v) a history of recurrent lung inflammation, (vi) a history of tuberculosis, (vi) a history of silicosis, berylliosis or talc inhalation, (vii) a family history of lung cancer in genetically related individuals, (viii) a history of vitamin A deficiency or vitamin A excess, (ix) a history of smoking cannabis, and, (ix) a history of exposure to one or more toxic volatile substances and/or to one or more infectious agents.

For background on these and other indicators of increased risk for developing lung cancer, see, e.g., Alberg et al., Chest 2003, 123:21 S-49S; U.S. Department of Health and Human Services, Health Consequences of Smoking: A Report of the Surgeon General (2004); Institute of Medicine (IOM) National Cancer Policy Board. Fulfilling the Potential of Cancer Prevention and Early Detection, Curry S J, Byers T, Hewitt M (eds), National Academies Press, Washington, D.C., 2003; National Institutes of Health/National Cancer Institute, Smoking Tobacco control monograph 9: Cigars, health effects and trends, NIH Publication No. 98-4302, Bethesda, Md., U.S. Department of Health and Human Services, 1998; Boffetta et al., Journal of the National Cancer Institute 1999, 91:697-701; National Research Council (NRC), Committee on Passive Smoking, Environmental Tobacco Smoke: Measuring Exposures and Assessing Health Effects (1986); U.S. Environmental Protection Agency, Respiratory Health Effects of Passive Smoking, (1992); International Agency for Research on Cancer (IARC), IARC Monographs on the Evaluation of Carcinogenic Risks to Humans and their Supplements, A complete list: Involuntary Smoking, Volume 83 (2002); International Agency for Research on Cancer (IARC), IARC Monographs on the Evaluation of Carcinogenic Risks to Humans and their Supplements, A complete list: Overall Evaluations of Carcinogenicity: An Updating of IARC Monographs Volumes 1 to 42. (1987); U.S. Department of Health and Human Services, Public Health Service, National Toxicology Program, Report on Carcinogens, Eleventh Edition (2004); International Agency for Research on Cancer (IARC), IARC Monographs on the Evaluation of Carcinogenic Risks to Humans and their Supplements, A complete list: Some Metals and Metallic Compounds and Arsenic and Arsenic Compounds, Volume 23 (1980); Etzel et al., Cancer Research 2003; 63:8531-8535; Brownson et al., International Journal of Epidemiology 1997; 26:256-263; Bromen et al., American Journal of Epidemiology 2000; 152:497-505; Mayne et al., Cancer Epidemiology, Biomarkers & Prevention 1999; 8:1065-1069; World Cancer Research Fund/American Institute for Cancer Research, Food, Nutrition and the Prevention of Cancer: A global perspective, Washington, D.C., American Institute for Cancer Research, 2007.

In preferred embodiments a biological fluid containing one or more antibodies is obtained from a subject (e.g., a pre-diagnostic subject) and contacted with at least one isolated pre-diagnostic lung cancer indicator protein, to detect the presence or absence in the biological fluid of an antibody that is capable of specifically binding to one or more of the pre-diagnostic lung cancer indicator proteins. Biological fluids are typically liquids at physiological temperatures and may include naturally occurring fluids present in, withdrawn from, expressed by or otherwise extracted from a subject or biological source (e.g., a human subject such as a pre-diagnostic subject or a patient, or a biological sample obtained directly or indirectly therefrom, such as a blood sample, biopsy specimen, tissue explant, organ culture or any other tissue or cell preparation from which an antibody-containing fluid can be prepared).

Certain biological fluids derive from particular tissues, organs or localized regions and certain other biological fluids may be more globally or systemically situated in a subject or biological source. Examples of biological fluids include blood, serum and serosal fluids, plasma, lymph, urine, cerebrospinal fluid, saliva, mucosal secretions of the secretory tissues and organs, vaginal secretions, ascites fluids such as those associated with non-solid tumors, fluids of the pleural, pericardial, peritoneal, abdominal and other body cavities, and the like. Biological fluids may also include liquid solutions contacted with a subject or biological source, for example, cell and organ culture medium including cell or organ conditioned medium, lavage fluids and the like. In certain highly preferred embodiments the biological sample is serum, and in certain other highly preferred embodiments the biological sample is plasma. In other preferred embodiments the biological sample is a cell-free liquid solution.

It is contemplated that in certain embodiments the antibodies are present in the biological fluid at the time of contacting with the pre-diagnostic lung cancer indicator protein(s), but the invention is not so limited and also contemplates embodiments in which the antibodies are isolated from the biological fluid prior to the step of contacting. The term “isolated” means that the material is removed from its original environment (e.g., the natural environment if it is naturally occurring). For example, a naturally occurring antibody (e.g., autoantibody), polypeptide or polynucleotide present in a living subject (e.g., a pre-diagnostic subject or a patient) is not isolated, but the same antibody, polypeptide or polynucleotide, separated from some or all of the co-existing materials in the natural system, is isolated. Such antibodies, polypeptides or polynucleotides could be part of a composition, and still be isolated in that such composition is not part of its natural environment.

Isolation of antibodies may be achieved according to any of a wide variety of methodologies with which persons skilled in the art will be familiar, including biochemical and/or immunological methods. Suitable biochemical techniques may include differential precipitation (e.g., as a function of salt or other solute concentration, for example, ammonium sulfate or sodium sulfate or polyethylene glycol (PEG) or the like), gel filtration chromatography, ion exchange chromatography, affinity chromatography (e.g., using lectin affinity or Staphylococcal protein A/protein G or mimetic affinity), hydrophobic interaction chromatography, chromatofocusing or isoelectric focusing (IEF) including free-fluid recycling IEF, high performance liquid chromatography (HPLC) or any of a number of other biochemical techniques such as well known separation techniques. Suitable immunochemical techniques include, but need not be limited to, immunoaffinity chromatography, immunoprecipitation, solid phase immunoadsorption or other immunoaffinity methods. For these and other useful techniques, see, for example, Scopes, R. K., Protein Purification: Principles and Practice, 1987, Springer-Verlag, NY; Weir, D. M., Handbook of Experimental Immunology, 1986, Blackwell Scientific, Boston; and Hermanson, G. T. et al., Immobilized Affinity Ligand Techniques, 1992, Academic Press, Inc., California.

The term “antibodies” includes immunoglobulins such as polyclonal antibodies, monoclonal antibodies, fragments thereof such as F(ab′)₂, and Fab fragments, as well as any naturally occurring immunoglobulin variable (V) region complementarity determining region (CDR)-containing binding partners (also including in certain embodiments antigen-binding CDR-containing T cell receptor polypeptides which are encoded by members of the immunoglobulin gene superfamily, whilst certain other embodiments expressly exclude such T cell-derived polypeptides), which are molecules that specifically bind a pre-diagnostic lung cancer indicator protein.

As described in greater detail below, particularly preferred embodiments relate to detection in a biological fluid from a pre-diagnostic subject of antibodies that are autoantibodies, i.e., antibodies that specifically recognize and bind to “self” antigenic epitopes that may also be found in the subject. Briefly, it is well accepted in the art that the immune system (e.g., adaptive immune system) is typically characterized as distinguishing foreign agents (or “non-self”) agents from familiar or “self” components, such that foreign agents elicit immune responses while “self” components are ignored or tolerated. Exceptions to this paradigm arise, however, in the case of autoantibody generation, whereby a host immune system produces antibodies that react with “self” antigens. See, e.g., Theofilopoulos and Bona, The Molecular Pathology of Autoimmune Diseases, CRC Press, Boca Raton, Fla., 2002; Doria et al., Handbook of Systemic Autoimmune Diseases (Vols. 1-9), Elsevier, N.Y., 2004-2008; Roitt et al., Immunology (6^{th} Ed.), Mosby, N.Y., 2001, Ch. 26. Accordingly and as described herein, there are provided the present embodiments in which an autoantibody is detected in the lung cancer diagnostic methods, wherein the autoantibody detectably and specifically binds to a pre-diagnostic lung cancer indicator protein, which protein may be a “self” component in the pre-diagnostic subject.

Antibodies are defined to be “immunospecific” or to be capable of specifically binding if they bind a pre-diagnostic lung cancer indicator protein (or a polypeptide that comprises one or more antigenic epitopes of such a protein) with a Kₐ of greater than or equal to about 10⁴ M⁻¹, preferably of greater than or equal to about 10⁵ M⁻¹, more preferably of greater than or equal to about 10⁶ M⁻¹ and still more preferably of greater than or equal to about 10⁷ M⁻¹.

Affinities of binding partners or antibodies can be readily determined using conventional techniques, for example those described by Scatchard et al., Ann. N.Y. Acad. Sci. 51:660 (1949), or by surface plasmon resonance (SPR) spectroscopy, or by any of a number of other known methods for identifying and characterizing antibodies or antibody-derived proteins that specifically interact with cognate antigens via recognition and binding of antigenic epitopes. For instance, to detect an antibody that specifically binds to a pre-diagnostic lung cancer indicator protein (or a polypeptide that comprises one or more antigenic epitopes of such a protein), there are a variety of assay formats, including but not limited to enzyme linked immunosorbent assay (ELISA), radioimmunoassay (RIA), immunofluorimetry, immunoprecipitation, equilibrium dialysis, immunodiffusion and other techniques. See, e.g., Harlow and Lane, Antibodies: A Laboratory Manual, Cold Spring Harbor Laboratory, 1988; Weir, D. M., Handbook of Experimental Immunology, 1986, Blackwell Scientific, Boston. See, e.g., Coligan et al. (Eds.), Current Protocols in Immunology (2007 John Wiley & Sons, NY); Harlow and Lane, Antibodies: A Laboratory Manual (1988 Cold Spring Harbor Press, Cold Spring Harbor, N.Y.); Harlow and Lane, Using Antibodies (1999 Cold Spring Harbor Press, Cold Spring Harbor, N.Y.). Therein can also be found parameters for designing immunoassay conditions, including conditions and incubation times that will be sufficient for detection of specific binding of an antibody to its cognate antigen.

### Pre-Diagnostic Lung Cancer Indicator Protein

As described herein, the present embodiments relate to detecting the presence in a biological fluid from a pre-diagnostic subject of an antibody that specifically binds to one or more isolated pre-diagnostic lung cancer indicator proteins. A pre-diagnostic lung cancer indicator protein thus may include any one or more of a number of target antigens for which the presence in a biological sample from a pre-diagnostic subject of a specifically reactive cognate antibody indicates that the subject has lung cancer. As is well known in the immunological art, specifically-binding antibodies define antigenic epitopes on their cognate antigens, i.e., the molecular structure with which the antibody's antigen-combining site interacts. For example, an antigenic epitope of a pre-diagnostic lung cancer indicator protein may be defined by a region of protein primary structure (e.g., amino acid sequence), by protein secondary structure (e.g., a localized structure that is spatially defined such as may be formed by a common motif or repetitive domain, e.g, alpha-helix, beta-sheet, etc.), by protein tertiary structure (e.g., by three-dimensional structure that is created by protein folding or conformation) or by quarternary structure (e.g., by the interaction of two or more polypeptide subunits, as in a complex or oligomer). Antigenic epitopes may additionally or alternatively be defined in whole or in part by post-translational modifications to pre-diagnostic lung cancer indicator proteins, including by virtue of direct involvement of the post-translational modification in formation of the epitope structure and also including, e.g., conformational epitopes the presence of which may depend on the presence of a particular post-translational modification. Accordingly, certain embodiments described herein contemplate use of an intact pre-diagnostic lung cancer indicator, which may bear one, two, three, four, five, six or more antigenic epitopes that can be defined by antibody reactivities, while certain other embodiments contemplate use of an isolated protein or polypeptide that comprises one or more antigenic epitopes of one or more pre-diagnostic lung cancer indicator proteins.

A pre-diagnostic indicator protein thus may be (i) a naturally occurring protein or polypeptide, (ii) a synthetic protein or polypeptide, (iii) a recombinant protein or polypeptide, or (iv) a fusion protein or polypeptide that comprises a fusion polypeptide domain fused to the pre-diagnostic indicator protein, or to the polypeptide that comprises one or more antigenic epitopes of the pre-diagnostic indicator protein. A pre-diagnostic indicator protein may be one or more of a non-posttranslationally modified protein, a posttranslationally modified protein, for example, a glycoprotein, a lipoprotein, a phosphoprotein, a proteolipid, a glycosylphosphatidylinositol-modified (“glypiated”) protein, a ubiquitinylated protein, a small ubiquitin-like modifier-modified (“SUMOylated”, Hay, 2005 Mol. Cell 18:1-12) protein, a sulfated protein and a glycated protein, and may (including in certain preferred embodiments) also be a posttranslationally modified protein in which one or more of the posttranslational modifications results in immunogenicity. Criteria and methodologies for distinguishing among these and other classes of post-translationally modified proteins (or unmodified proteins) will be known to those familiar with the relevant art, as also will be methodologies for determining whether immunogenicity, including for example the formation of one or more particular antibody—(e.g., autoantibody) defined epitopes, resides in the structure formed by a posttranslational modification (e.g., Ahmed, Principles and Reactions of Protein Extraction, Purification, and Characterization, Taylor & Francis, NY, 2007; Scopes, R. K., Protein Purification: Principles and Practice, 1987, Springer-Verlag, NY; Coligan et al. (Eds.), Current Protocols in Immunology (2007 John Wiley & Sons, NY)).

Based on the disclosure herein and knowledge in the art concerning the making and testing of synthetic, recombinant and/or fusion polypeptides and proteins, it will also be appreciated that through routine methodologies non-naturally occurring polypeptides and proteins can be constructed and immunologically (and/or structurally) probed for the presence of antibody-reactive epitopes. Exemplary methodologies may be found, for example, in e.g., Bonificano et al. (Eds.) Current Protocols in Cell Biology, 2007 John Wiley & Sons, NY; Ausubel et al. (Eds.) Current Protocols in Molecular Biology, 2007 John Wiley & Sons, NY; Coligan et al. (Eds.), Current Protocols in Immunology, 2007 John Wiley & Sons, NY; Robinson et al. (Eds), Current Protocols in Cytometry, 2007 John Wiley & Sons, NY. For instance, an antigenic epitope that can be specifically recognized by an autoantibody from a pre-diagnostic subject having lung cancer may be a truncated pre-diagnostic lung cancer indicator protein such as a functional fragment thereof, e.g., a portion of the protein that retains the antigenic epitope as can be readily determined by immunological cross-reactivity with the epitope-bearing, full length intact protein.

As also noted above, according to non-limiting theory the presence of lung cancer induces generation by the host immune system of antibodies reactive with the herein described pre-diagnostic lung cancer indicator protein(s), even in a pre-diagnostic subject, i.e., at a time when lung cancer is undetectable in the subject by any diagnostic means of the prior art, including in the absence of any signs or symptoms of lung cancer in the subject. Without wishing to be bound by theory, it is contemplated that certain pre-diagnostic lung cancer indicator protein-reactive antibodies (including in preferred embodiments autoantibodies) are generated by the pre-diagnostic subject's immune system in response to immune recognition of one or more pre-diagnostic lung cancer indicator proteins that may be expressed by lung cancer cells and/or by precancerous cells that are inexorably committed to progression into lung cancer cells and/or by other cells associated with such lung cancer or precancerous cells. The invention embodiments need not, however, be so limited, and also contemplate certain other pre-diagnostic lung cancer indicator protein-reactive antibodies (including in preferred embodiments autoantibodies) the production of which in the subject may be elicited by aberrant immune function, or serendipitously by cross-reactive antigens to give rise to heteroclitic antibodies (e.g., antibodies that react more strongly with antigens other than those used to elicit their production).

As described in greater detail below, certain particularly preferred embodiments relate to diagnostic and screening methods in which the pre-diagnostic lung cancer indicator protein is the laminin receptor precursor protein known as LAMR1, which may be any one of a set of related protein isoforms (including allelotypes) believed to be encoded by a common gene and identified in several different forms that differ in molecular weight, oligomeric state, post-translational modification, supramolecular associations, cell source and presumed function, as described below. For example, LAMR1 proteins include the 295 amino acid, 33 kDa laminin receptor precursor, and may also occur as a ribosomal 40S subunit-associated protein, and may also occur as an oncofetal antigen and/or as a eukaryotic cell prion receptor (e.g., accession numbers IP100411639.1; IP100413108.4; IP100553164.4; IP100790580.1; IP100793905.1; SEQ ID NOS:6-10).

In certain related embodiments one or both of the pre-diagnostic lung cancer indicator proteins Annexin 1 and 14-3-3 theta protein may also be used. Annexin proteins including annexin I may have variable posttranslationally added glycosylation patterns, which may contribute to immunogenicity of the resulting glycoproteins. Annexin 1 proteins thus include glycoforms and other isoforms (and further include allelotypes) of the cytoplasmic calcium-dependent phospholipid-binding annexin I member of the annexin family of conserved proteins that is widely expressed in eukaryotic cells (e.g., accession numbers IP100218918.5; IP100549413.2; IP100643231.1; SEQ ID NOS:1-3; see also, e.g., Brichory et al., 2001 Proc. Nat. Acad. Sci. USA 98:9824; U.S. Pat. No. 6,645,465).

14-3-3 theta proteins include isoforms (and further include allelotypes) of the 14-3-3 theta gene products, which are embers of the widely expressed 14-3-3 protein family involved in signal transduction and cell cycle control; 14-3-3 theta proteins also include phosphorylated, acetylated, cleaved, and truncated variants (see, e.g., Pereira-Faca et al., 2007 Canc. Res. 67:12000). Exemplary sequences are set forth below (e.g., accession numbers IP100018146.1; IP100796727.1; SEQ ID NOS:4-5).

In certain related embodiments one, two, three, four, five, six, seven, eight or more of the pre-diagnostic lung cancer indicator proteins AKR1B10 protein [SEQ ID NO:11], GOT2 protein [SEQ ID NO:12], HNRPR protein [SEQ ID NO:13], PDIA3 protein [SEQ ID NO:14], NME2 protein [SEQ ID NO:15], RTN4 protein [SEQ ID NO:16], HI1FX protein [SEQ ID NO:17], G3BP protein [SEQ ID NO:18], HSPCA protein [SEQ ID NO:19], and ACTN4 protein [SEQ ID NO:20] may, additionally or alternatively, be used. (See, e.g., Table 5.)

As known in the art “similarity” between two polypeptides is determined by comparing the amino acid sequence and conserved amino acid substitutes thereto of the polypeptide to the sequence of a second polypeptide. Similarity between two polypeptide (or encoding polynucleotide) sequences, or even the percent identity, may be readily determined by comparing sequences using computer algorithms well known to those of ordinary skill in the art, such as the BLAST algorithm (Altschul, J. Mol. Biol. 219:555-565, 1991; Henikoff and Henikoff, Proc. Natl. Acad. Sci. USA 89:10915-10919, 1992), which is available at the NCBI website (http://www/ncbi.nlm.nih.gov/cgi-bin/BLAST). Default parameters may be used. Examples of other useful computer algorithms are those used in programs such as Align and FASTA, which may be accessed, for example, at the Genestream internet website of the Institut de Genetique Humaine, Montpellier, France (www2.igh.cnrs.fr/home.eng.html) and used with default parameters. Fragments or portions of the pre-diagnostic lung cancer indicator proteins or polypeptides derived therefrom may be employed in certain herein disclosed embodiments, which fragments or portions retain at least one antigenic epitope that is capable of being specifically recognized by an antibody from a biological fluid of a pre-diagnostic subject having lung cancer, and which may have at least 50, 60, 70, 80, 85, 90, 91, 92, 93, 94, 95, 96, 97, 98, or 99 percent identity to the herein disclosed pre-diagnostic lung cancer indicator protein sequences.

The methods to be practiced according to certain herein disclosed embodiments may involve any convenient format for interrogating a biological fluid, as provided herein, for the presence of one or more antibodies that are capable of specifically binding to a pre-diagnostic lung cancer indicator protein as provided herein. Alternatively, certain embodiments may involve any convenient format for interrogating an antibody-containing fraction that has been isolated from a biological fluid, as provided herein, for the presence of one or more antibodies that are capable of specifically binding to a pre-diagnostic lung cancer indicator protein as provided herein.

As also noted above, any of a number of convenient formats may be employed for contacting one or more antibodies from a pre-diagnostic biological fluid with an isolated pre-diagnostic lung cancer indicator protein to detect specific antibody binding to the indicator protein, including but not limited to enzyme linked immunosorbent assay (ELISA), surface plasmon resonance (SPR) spectroscopy, western blot immunoassay, radioimmunoassay (RIA), immunofluorimetry, immunoprecipitation, equilibrium dialysis, immunodiffusion and other techniques. See, e.g., Harlow and Lane, Antibodies: A Laboratory Manual, Cold Spring Harbor Laboratory, 1988; Weir, D. M., Handbook of Experimental Immunology, 1986, Blackwell Scientific, Boston. See, e.g., Coligan et al. (Eds.), Current Protocols in Immunology (2007 John Wiley & Sons, NY); Harlow and Lane, Antibodies: A Laboratory Manual (1988 Cold Spring Harbor Press, Cold Spring Harbor, N.Y.); Harlow and Lane, Using Antibodies (1999 Cold Spring Harbor Press, Cold Spring Harbor, N.Y.).

Briefly, for example by way of illustration and not limitation, one or more isolated pre-diagnostic lung cancer indicator proteins (or polypeptides comprising at least one antigenic epitope of a pre-diagnostic lung cancer indicator protein) may be immobilized on a solid-phase substrate, which may be non-covalent immobilization (e.g., by passive adsorption to the solid phase) or covalent immobilization (e.g., by chemically crosslinking the protein or polypeptide to the solid phase using any of a number of well known functional groups). The biological fluid from a pre-diagnostic subject is then contacted with the solid phase, under conditions and for a time sufficient for specific binding of at least one antibody from the fluid to be detected, which may typically include an incubation period to permit such binding followed by a washing step to remove any non-specifically binding components of the biological fluid. The specifically bound antibody may then be detected by any of a number of means for detecting, for example, by mass spectrometric shift if SPR is used, or as another example, by further contacting the solid phase with a detectably labeled secondary (or “second stage”) antibody such as an anti-immunoglobulin (e.g., a rabbit anti-human immunoglobulin) followed, after a suitable incubation period and conditions with which the skilled artisan will be familiar, by another washing step to remove unbound secondary antibody, after which the detectable label may be detected and compared to one or more appropriate controls for purposes of determining whether an antibody that specifically binds to a pre-diagnostic lung cancer indicator protein is present. It will be appreciated that other assay techniques, formats and configurations may also be employed. As described herein, detection of an antibody, in a biological fluid from a pre-diagnostic subject, that specifically binds to a pre-diagnostic lung cancer indicator protein as provided herein, indicates that the pre-diagnostic subject has lung cancer.

Accordingly and in certain embodiments, detection of antibody binding to a pre-diagnostic lung cancer indicator protein (or to a polypeptide that comprises one or more antigenic epitopes therefrom) may comprise detection using a specific, detectably labeled secondary reagent (such as an anti-immunoglobulin antibody, or Staphylococcal protein A or protein G or an immunologically active fragment thereof or a mimetic thereof that specifically binds a human immunoglobulin constant region) that contains a detectable reporter moiety or label such as an enzyme, dye, radionuclide, luminescent group, fluorescent group or biotin, or the like. The amount of the detectably labeled secondary reagent that remains bound to the pre-diagnostic lung cancer indicator protein (or to the polypeptide that comprises one or more antigenic epitopes therefrom) is then determined using a method appropriate for the specific detectable reporter moiety or label. For radioactive groups, scintillation counting or autoradiographic methods are generally appropriate. Antibody-enzyme conjugates may be prepared using a variety of coupling techniques (for review see, e.g., Scouten, W. H., Methods in Enzymology 135:30-65, 1987). Spectroscopic methods may be used to detect dyes (including, for example, colorimetric products of enzyme reactions), luminescent groups and fluorescent groups. Biotin may be detected using avidin or streptavidin, coupled to a different reporter group (commonly a radioactive or fluorescent group or an enzyme). Enzyme reporter groups may generally be detected by the addition of substrate (generally for a specific period of time), followed by spectroscopic, spectrophotometric or other analysis of the reaction products.

In addition to providing embodiments that find uses as methods for diagnosing lung cancer and/or screening methods for lung cancer, e.g., convenient testing of biological fluids from a plurality of subjects such as a population of subjects that may be at increased risk for developing lung cancer, for instance, by virtue of having one or more indicators of increased risk for developing lung cancer as described above, other embodiments contemplated herein relate to a method of monitoring lung cancer autoimmune reactivity in a lung cancer patient. According to these and related embodiments, a subject that has already been diagnosed with lung cancer may exhibit qualitative and/or quantitative changes in the antibodies that are present in biological fluid at different points in time.

For instance, such changes from a first timepoint to a second timepoint may reflect progression of the disease. As another example, antibody reactivity against one or more pre-diagnostic lung cancer indicator proteins (or polypeptides comprising epitopes derived therefrom) may be tested at one or a plurality of timepoints before, during or after administration to the lung cancer patient of a therapeutic agent. Detection of autoantibody binding over time may therefore provide a means for monitoring the cancer status of the patient, such as indicating whether the patient is responding to therapy, and/or whether the patient may be in remission or may be relapsing. Determination of quantitative and qualitative changes over time in lung cancer indicator protein-reactive antibodies as disclosed herein is within the capability of the art, for instance, by using established immunochemical methodologies for assaying the amount of autoantibody in a patient's biological fluid, or the affinities of autoantibodies in the biological fluid, or a change in the isotypes of the autoantibodies, or a change in the antigen- and/or epitope specificities of the antibodies (e.g., autoantibodies) that are detected in the cancer patient's biological fluid.

Accordingly, there is provided in certain representative embodiments a method of monitoring lung cancer autoimmune reactivity in a lung cancer patient, comprising contacting, after each of two or more timepoints, (i) one or more antibodies from a biological fluid that is taken from a subject at each of said timepoints, and (ii) a test antigen that may be one or more isolated pre-diagnostic lung cancer indicator proteins, or that may instead be one or more isolated proteins or polypeptides that comprise one or more antigenic epitopes of one or more pre-diagnostic lung cancer indicator proteins, under conditions and for a time sufficient for detecting specific binding of at least one antibody from the biological fluid to one or more of the pre-diagnostic lung cancer indicator proteins or antigenic epitopes thereof; and comparing the specific binding that is detectable by antibodies from the biological fluid taken at each of the two or more timepoints, and thereby monitoring lung cancer autoimmune reactivity in the patient. Two, three, four, five, six, seven, eight, nine, ten or more timepoints may be identified in order to practice these and related embodiments, as may be indicated for the appropriate monitoring of the lung cancer patient's autoimmune reactivity. For instance, a first timepoint may occur before administration of a therapeutic agent to the patient, and the second and subsequent timepoints may occur after administration of the therapeutic agent to the patient.

It is also contemplated according to certain other embodiments based on the disclosure made herein that the present compositions and methods may permit subtyping of a tumor in a subject (including but not limited to a pre-diagnostic subject), and may also permit subtyping of a plurality of patients, on the basis of the qualitative and/or quantitative autoantibody reactivity that can be detected against one or more of the pre-diagnostic lung cancer indicator proteins as provided herein. Thus, for example, accumulation of data obtained according to the herein disclosed methods may lead to more specific diagnostic information beyond the detection of the presence of lung cancer, such as the degree of progression or other qualitative and/or quantitative criteria by which the cancer can be characterized.

Additionally or alternatively, it is contemplated that detection of one or more autoantibodies specific for one or more of the pre-diagnostic lung cancer indicator proteins as provided herein may usefully define the histological subtype of one or more given lung cancers. For example, a lung cancer detected according to the present disclosure may be a primary tumor originating in the lung, or may instead be a lung cancer that results from metastasis to the lung of cancer cells originating in a distinct anatomical site, such as breast carcinoma, colorectal carcinoma, head and neck squamous cell carcinoma, gastrointestinal carcinoma, testicular cancer or any number of other potential primary tumors having distinct sites of origin. Information obtained through the practice of these and related embodiments may provide the clinician with important data for the formulation of an effective therapeutic strategy, given that tumors of different origins may respond differentially (or not at all) to different anti-cancer treatment regimens.

Application of the herein described proteomics approach for the identification of additional pre-diagnostic lung cancer indicator proteins, is also contemplated.

The following Examples are presented by way of illustration and not limitation.

### EXAMPLES

In the present Examples, pre-diagnostic sera were utilized to determine if a set of antigens consisting of annexin I, PGP9.5 and 14-3-3 theta previously found to be associated with autoantibodies at the time of diagnosis discriminate between cases and control prior to onset of symptoms, and for discovery of additional antigens. Evidence is presented for the occurrence of autoantibodies against a novel antigen, LAMR1, in lung cancer, along with evidence for occurrence of autoantibodies against annexin I, 14-3-3 theta and LAMR1 in pre-diagnostic sera.

A high throughput platform for quantitative analysis of serum autoantibodies was applied to lung cancer for discovery of novel antigens, and for validation in pre-diagnostic sera of autoantibodies to antigens previously defined based on analysis of sera collected at the time of diagnosis.

Proteins from human lung adenocarcinoma cell line A549 lysates were subjected to extensive fractionation. The resulting 1824 fractions were spotted in duplicate on nitrocellulose coated slides. The microarrays produced were utilized in a blinded validation study to determine whether annexin I, PGP9.5, and 14-3-3 theta antigens previously found to be targets of autoantibodies in newly diagnosed subjects with lung cancer were associated with autoantibodies in sera collected at the pre-symptomatic stage and to determine whether additional antigens may be identified in pre-diagnostic sera. Individual sera collected from 85 subjects within a year prior to a diagnosis of lung cancer and 85 matched controls from the CARET cohort were hybridized to individual microarrays.

Evidence is presented herein for the occurrence in pre-diagnostic lung cancer sera of autoantibodies to annexin I, 14-3-3 theta, and a novel lung cancer antigen, LAMR1. Detectable appearance of these autoantibodies preceded onset of lung cancer symptoms and also preceded diagnosis of lung cancer by any other means. Diagnosis of lung cancer before onset of symptoms is thus described herein, by screening for autoantibodies specific for pre-diagnostic lung cancer indicator proteins as the defined antigens.

### Example 1

### Materials and Methods

### Materials

Nitrocellulose-coated FAST slides were purchased from Whatman (Sanford, Me.). Alexa 647-labeled anti-human IgG and recombinant protein arrays were purchased from Invitrogen (Carlsbad, Calif.).

### Serum Samples

Serum samples and controls were obtained following informed consent. Sera from newly diagnosed lung cancer patients and matched controls were collected through the Community Clinical Oncology Program at the University of Michigan. Pre-diagnostic blood samples from lung cancer patients and matched controls were randomly chosen in pairs from the CARET serum bank (Goodman et al., J Natl Cancer Inst 96:1743-50, 2004; Omenn et al., N Engl J Med 334:1150-5, 1996). The distribution of histology and time from blood draw to diagnosis for the 85 pre-diagnostic lung cancer cases are shown in Tables 1 and 2.

Natural Protein Microarray Production

50 mg proteins from the human lung adenocarcinoma cell line A549 lysates were first separated by anion exchange HPLC, followed by reverse-phase chromatography as described previously (Wang and Hanash, J Chromatogr B Analyt Technol Biomed Life Sci 787:11-8, 2003). A total of 1,824 fractions were collected from the 2-dimensional separation. FR_XX_YY denotes the YYth fraction from the RP-HPLC of the XXth fraction from the AEX. Fractions were lyophilized and re-suspended in 25 ul printing buffer (250 mM Tris-HCl, pH 6.8, 0.5% SDS, 25% Glycerol, 0.05% TritonX-100, 62.5 mM DTT). All 1,824 fractions, together with printing buffer as negative control and purified human IgG as positive controls, were printed in duplicate onto nitrocellulose-coated slides using a contact printer. These slides were designated as “full A549 natural protein microarrays.” An A549 natural protein microarray containing selected fractions (targeted array) was produced in a similar way, whereby only selected fractions of interest (Fr_{—}00_{—}84, Fr_{—}09_{—}38, Fr 15_{—}39 and Fr 15_{—}46) were printed in duplicate on 16-pad FAST slides.

Detection of Autoantibodies in Serum Specimens

Serum samples were hybridized with protein microarrays using an indirect immunofluorescence protocol and local background subtracted median spot intensities for downstream statistical analysis were generated as described previously (Qiu et al., J Proteome Res 3:261-7, 2004).

Mass-Spectrometry Analysis

Samples were trypsin-digested and subjected to mass spectrometry analysis on an LTQ-Orbitrap as described previously (Faca et al., J Proteome Res 6:3558-65, 2007).

Statistical Analysis

Intensity data were linear normalized to make the 25th and 75th percentiles of the distribution of the intensities for each sample agree exactly with the average of the 25th and 75th percentiles of all samples by linear transformations. Linear normalized data were then standardized fraction-by-fraction. Each sample/fraction intensity was subtracted off the mean of the same fraction for all samples in the same printing batch and divided by the standard deviation of the fraction for all samples in the corresponding printing batch. A two-sample t-test was applied to each single fraction to compare the difference in mean intensity between cancers and controls.

The discriminatory capacity of selected fractions was also evaluated by the Receiver Operating Characteristic (ROC) curve. The area under the curve (AUC) was calculated which corresponds to the Mann-Whitney statistic. Parallel analyses using a generalized version of the “real boosting” algorithm with 10-fold cross-validation was also performed on the 1824 fractions to select the best combination of fraction(s) that can discriminate between cases and controls (Friedman et al., Annals of Statistics 28:337-407, 2000; Yasui et al., Biostatistics 4:449-63, 2003). The results were treated as part of a separate biomarker discovery process. Even if a promising fraction was identified as a previously-identified antigen associated with lung cancer, it was treated as an independent confirmation of the previous findings rather than as part of the validation study because that antigen had not been established for validation at the design stage of the study.

For combined analysis, the markers were integrated by a summation of the dichotomized markers, whereby each marker was dichotomized by its optimal cutoff point which corresponds to the minimum classification error rate. The 95% confidence interval (CI) band of the ROC curve was estimated from 500 bootstrap procedures. The combination rule was treated as a “discovered biomarker” and was regarded as amenable to further validation; however, it provided information on the complementarities of informative antigens.

### Example 2

### Results

Validation Study of Autoantibodies to Annexin I, PGP9.5, and 14-3-3 Theta in Pre-Diagnostic Sera

Annexin I, PGP9.5 and 14-3-3 theta were previously identified as inducing an autoantibody response in lung cancer, based on 2D Western analysis of sera from newly diagnosed subjects with lung cancer (Brichory et al., Cancer Res 61:7908-12, 2001; Brichory et al., Proc Natl Acad Sci USA 98:9824-9, 2001; Pereira-Faca et al., Cancer Res 67:12000-6, 2007). Natural protein microarrays were developed to screen tumor-derived proteins for antigens that induce autoantibodies, based on extensive protein fractionation followed by spotting of aliquots from individual fractions (Madoz-Gurpide et al., Proteomics 1:1279-87, 2001). Natural protein containing microarrays were utilized to investigate the occurrence of autoantibodies to annexin I, PGP9.5 and 14-3-3 theta reactivity in pre-diagnostic sera. Serum specimens from the CARET cohort, which consisted of subjects at increased risk for lung cancer followed longitudinally (Omenn et al., N Engl J Med 334:1150-5, 1996), were relied upon to investigate the occurrence of autoantibodies to lung cancer antigens within a year prior to diagnosis. Each case and control pair was matched for age at enrollment (5-year intervals), sex, intervention arm (active vitamins or placebo), exposure population (asbestos or heavy smoker), baseline smoking status (active or former), year of enrollment, and year of blood draw. Eighty-five pre-diagnostic lung cancer specimens and an equal number of matched controls were utilized for this study (Tables 1, 2).

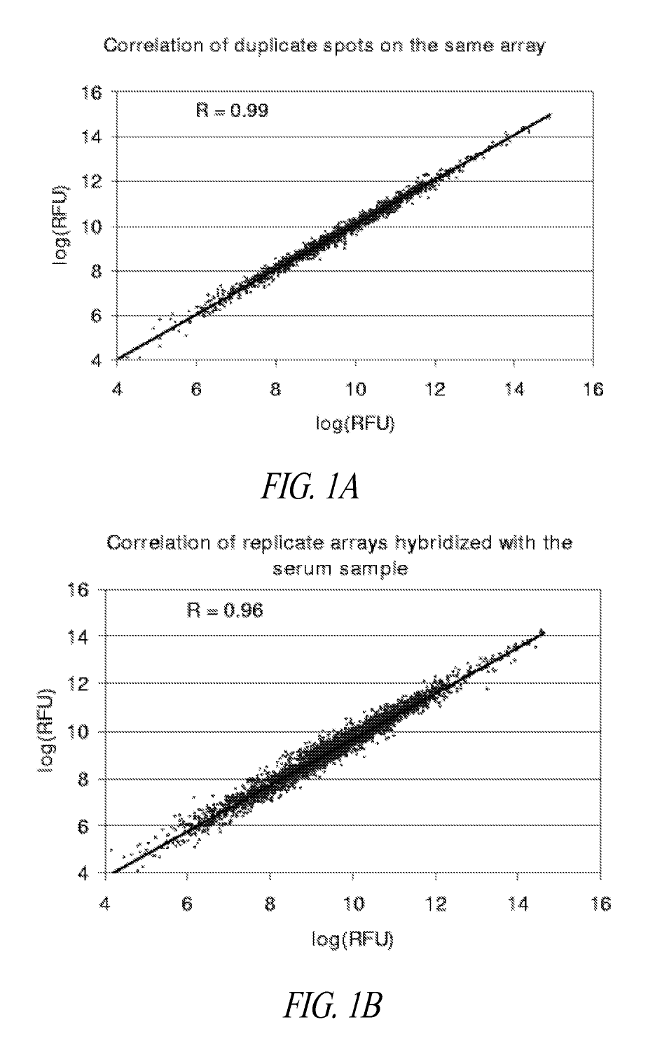

A549 natural protein microarrays were prepared from 2-D separations of a batch of A549 cell lysates. Quantitative reproducibility of microarrays was assessed by replicate analysis. Reproducibility across microarrays was assessed by hybridization of the same sample on different microarrays. Reproducibility within slides was assessed by replicate spots on the same microarray. Correlation of replicate spot intensity measures in the same microarray was 0.99. (FIG. 1A) Correlation of spot intensity measures between different microarrays hybridized with the same sera was 0.96 (FIG. 1B). The median IgG reactivity for cancer samples, across the entire spotted array, was similar to that for normal controls (data not shown). However the number of fractions with significant p-values <0.05 with measures for cancer greater than for control was higher than the number of fractions for which control was greater than cancer. Of 1824 arrayed fractions, 68 fractions gave a p-value <0.05 with mean intensity for cancer greater than control, while there were only 16 fractions with a p-value <0.05 with mean intensity for controls greater than for cancer.

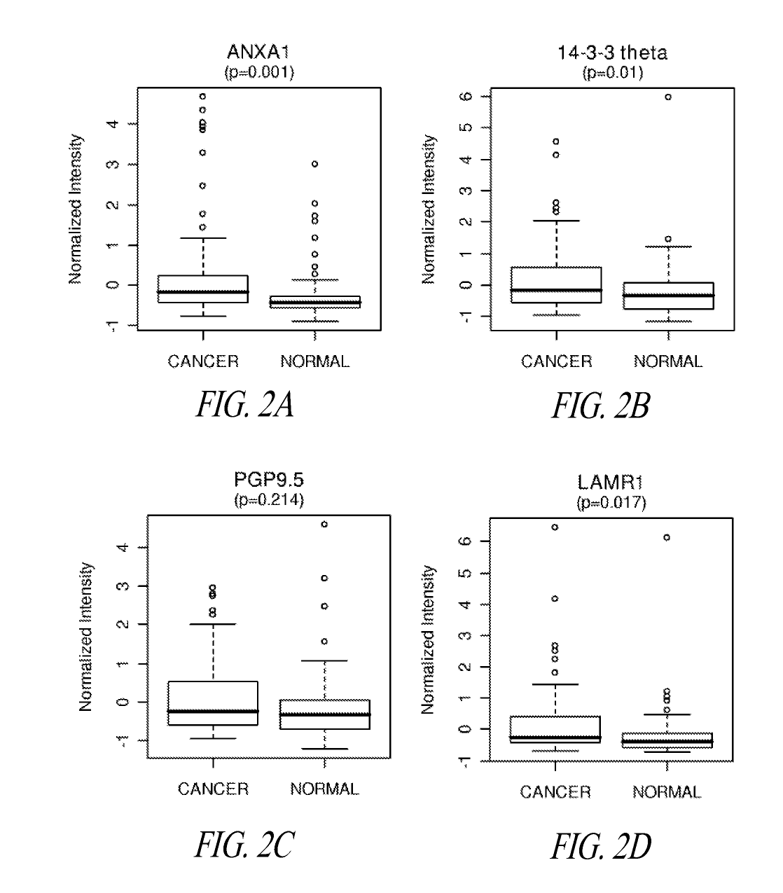

Annexin I was localized on the A549 natural protein microarrays in Fr_{—}00_{—}84. PGP9.5 was localized in Fr_{—}09_{—}38 and 14-3-3 theta was localized in Fr_{—}15_{—}46 based on reactivity with corresponding monoclonal antibodies. Following blinded analysis of all sera, the pre-diagnostic cases had significantly higher mean annexin I autoantibody levels than those of controls (t-test p-value=0.001) (FIG. 2A). The p-value for 14-3-3 theta was also significant at 0.01 (FIG. 2B). On the other hand, the average PGP9.5 autoantibody reactivity of pre-diagnostic cases was not significantly different from that of matched high-risk controls from CARET (p=0.2) (FIG. 2C). These results were in concordance with prior results based on 2-D Western analysis of 18 of the 85 CARET pre-diagnostic sera used in the present study (Pereira-Faca et al, Cancer Res 67:12000-6, 2007).

Differential Reactivity to LAMR1 in Pre-Diagnostic Lung Cancer Sera

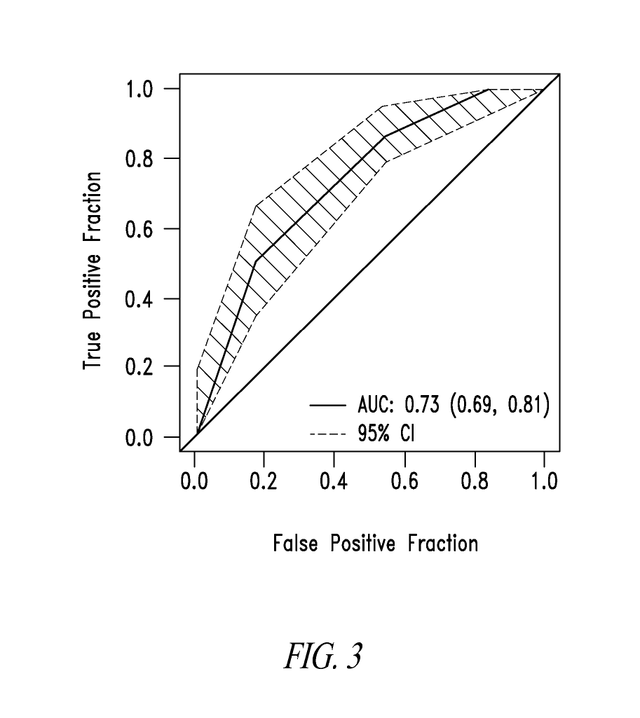

A boosting logistic regression method with leave-ten-percent-out for cross-validation was initially utilized to determine if additional arrayed proteins exhibited differential reactivity with pre-diagnostic cases relative to control sera. A spotted fraction (Fr_{—}15_{—}39), which was among the most informative in each of ten iterations of the model building procedure, yielded LAMR1 protein identification with high confidence by mass spectrometry based on mass spectral matching to 21 unique peptides in the LAMR1 sequence (65% coverage). The mean level of autoantibodies against LAMR1 in pre-diagnostic lung cancer sera was significantly higher than that in matched controls with a p-value of 0.017 (FIG. 2D). The combined AUC for all three antigens, annexin I, 14-3-3 theta and LAMR1, in CARET pre-diagnostic sera vs. matched controls was 0.73 (FIG. 3). The sensitivity and specificity at the optimal cutoff point was 51% and 82%, respectively.

CARET lung cancer cases represented three groups, adenocarcinoma (AC), squamous cell carcinoma (SCC), and other non-small cell lung cancer (NSCLC) (Table 1). An ANOVA test was performed to determine correlation between autoantibody levels against annexin I, PGP9.5, 14-3-3 theta, and LAMR1 and lung cancer subtype. Annexin I, PGP9.5 and LAMR1 did not show significant reactivity difference among the three subtypes. A significant p-value of 0.007 was obtained for 14-3-3 theta, with the AC and the SCC groups exhibiting lesser reactivity than other NSCLC. CARET cancer sera analyzed were collected within one year prior to diagnosis (Table 2). A possible relationship was investigated between autoantibody levels and the time from blood draw to diagnosis, by stratifying the samples into two groups, one group with cases collected between 0-6 months (inclusive) prior to diagnosis and the other group between 7-12 months (inclusive) prior to diagnosis. The mean reactivity for cases in the 0-6 group was higher than that for cases in the 7-12 group with t-test p-values of 0.05, 0.06 and 0.07 for PGP9.5, 14-3-3 theta and LAMR1 respectively. There was equivalent reactivity between the two groups for annexin 1 (Table 3). When comparing cases in each group against matched controls, the differences were also more significant in the 0-6 group than in the 7-12 group for PGP9.5, 14-3-3 theta and LAMR1 (Table 4).

Assessment of Autoantibody Reactivity to LAMR1 in Sera from Newly-Diagnosed Lung Cancer Subjects

The occurrence of autoantibodies to LAMR1 in sera from newly diagnosed subjects with lung cancer was determined using spotted microarrays containing purified recombinant LAMR1 that were reacted with sera from 45 newly diagnosed subjects with lung cancer and from an equal number of healthy controls that were matched for age and gender and time of blood collection. Increased LAMR1 reactivity among lung cancer sera relative to controls was observed based on two-sample t-test (p-value=0.024). Most of the lung cancer cases (32/45) had adenocarcinoma. A significant p-value of 0.03 was also obtained for the 32 subjects with adenocarcinoma relative to matched controls.

### Discussion

The results indicated that autoantibodies against LAMR1, annexin I and 14-3-3 theta were significantly elevated in pre-clinical lung cancer patient sera compared with matched high risk controls that did not develop lung cancer during the period of follow-up. A combination of LAMR1, annexin 1 and 14-3-3 theta autoantibodies yielded an AUC of 0.73 in pre-clinical lung cancer sera. While autoantibodies to various cancer antigens have been reported in newly diagnosed lung cancer patient sera (Stockert et al., J Exp Med 187:1349-54, 1998; Gure et al., Cancer Res 58:1034-41, 1998; Yamamoto et al., Int J Cancer 69:283-9, 1996; Diesinger et al., Int J Cancer 102:372-8, 2002; Gure et al., Proc Natl Acad Sci USA 97:4198-203, 2000; Ali Eldib et al., Int J Cancer 108:558-63, 2004; Yang et al., J Proteome Res 6:751-8, 2007; Brichory et al., Cancer Res 61:7908-12, 2001; Brichory et al., Proc Natl Acad Sci USA 98:9824-9, 2001; He et al., Cancer Sci 98:1234-40, 2007; Tureci et al., Cancer Lett 236:64-71, 2006; Chang et al: FEBS Lett 579:2873-7, 2005; Yagihashi et al: Lung Cancer 48:217-21, 2005; Matsumoto et al: Int J Oncol 19:1035-9, 2001; Fernandez-Madrid et al: Clin Cancer Res 5:1393-400, 1999; Lubin et al: Nat Med 1:701-2, 1995), a distinguishing feature of the present disclosure was the testing for occurrence of autoantibodies in pre-diagnostic sera and demonstration of significant autoantibody reactivity against LAMR1, annexin 1 and 14-3-3 theta. The sample size, 85 cases and an equal number of controls, and the characteristics of controls, heavy smokers or subjects who have been exposed to asbestos, were important features of the present study.

Autoantibodies against annexin I, PGP9.5 and 14-3-3 theta have previously been reported in newly diagnosed lung cancer patients (Brichory et al., Cancer Res 61:7908-12, 2001; Brichory et al., Proc Natl Acad Sci USA 98:9824-9, 2001). The data presented herein validated the occurrence of autoantibodies to annexin 1 and 14-3-3 theta, by demonstrating their presence also in pre-diagnostic lung cancer sera using natural protein arrays, yielding significant p-values of 0.001 and 0.01 respectively for differences with matched controls.

The present application discloses for the first time the occurrence of autoantibodies to LAMR1 in lung cancer.

The full length LAMR1 gene encodes a 33 kD precursor protein with 295 amino acids (Yow et al., Proc Natl Acad Sci USA 85:6394-8, 1988). Its precursor and post-translationally modified forms serve diverse biological functions in vivo. The 33 kD precursor protein dimerizes after acylation to form the mature 67LR (Buto et al., J Cell Biochem 69:244-51, 1998), which was initially purified using affinity chromatography on Sepharose® columns conjugated with laminin and designated as the 67 kD laminin receptor (67LR) (Rao et al., Biochemistry 28:7476-86, 1989; Lesot et al., EMBO J 2:861-865, 1983; Malinoff et al., J Cell Biol 96:1475-9, 1983).

The 33 kD precursor is an evolutionarily conserved ribosomal protein associated with the 40S subunit of the translational machinery (Auth et al., Proc Natl Acad Sci USA 89:4368-72, 1992; Ardini et al., Mol Biol Evol 15:1017-25, 1998). A 44 kD protein originally identified as an oncofetal antigen by Coggin et al. was subsequently found to be encoded by the same gene as 67LR (Coggin et al., Anticancer Res 19:5535-42, 1999; Coggin et al., Immunol Today 19:405-8, 1998). The precursor also serves as the receptor for the prion protein in eukaryotic cells (Rieger et al., Nat Med 3:1383-8, 1997).

67LR plays a role in cancer invasion and metastasis, related to its high affinity to laminin, an important component of basement membrane (Wewer et al., Cancer Res 47:5691-8, 1987). Over-expression of 67LR has been observed in melanomas, lymphomas and epithelial tumors (Menard et al., Breast Cancer Res Treat 52:137-45, 1998; Menard et al., J Cell Biochem 67:155-65, 1997; Cioce et al., J Natl Cancer Inst 83:29-36, 1991). Expression of 67LR correlates with poor prognosis in non-small cell lung cancer (Fontanini et al., Clin Cancer Res 3:227-31, 1997). There is evidence that the monomeric membrane-associated 44 kD OFA/iLRP (oncofetal antigen/immature laminin protein), but significantly, not 67LR, is immunogenic (Coggin et al., Anticancer Res 19:5535-42, 1999), consistent with the findings disclosed herein of autoantibodies in lung cancer. OFAs are expressed in fetal cells and a variety of cancers but not present in normal neonatal or adult tissues (Coggin et al., Immunol Today 19:405-8, 1998).

Immunization of adult hamsters with irradiated fetal hamster or mouse cells provides strong immunity to SV40-induced tumorigenesis (Ambrose et al., Nature 233:194-5, 1971; Coggin et al., J Immunol 107:526-33, 1971; Ambrose et al., Nature 233:321-4, 1971). OFA/iLRP was later identified as the protective antigen on the membrane of rodent and human fetal and tumor cells (Coggin et al., Anticancer Res 19:5535-42, 1999; Payne et al., J Natl Cancer Inst 75:527-44, 1985; Coggin et al., Am J Pathol 130:136-46, 1988; Gussack et al., Cancer 62:283-90, 1988). OFA/iLRP studies have largely focused on cellular immunity and its potential utility in T-cell based immunotherapy (Rohrer et al., J Immunol 152:754-64, 1994; Siegel et al., J Immunol 176:6935-44, 2006; Rohrer et al., J Immunol 154:2266-80, 1995; Holtl et al., Clin Cancer Res 8:3369-76, 2002; Rohrer et al., J Immunol 155:5719-27, 1995; Rohrer et al., J Immunol 176:2844-56, 2006; Rohrer et al., J Immunol 162:6880-92, 1999).

Although a humoral immune response could be induced in mice immunized with recombinant OFA/iLRP (Rohrer et al., Mod Asp Immunobiol 1:191-5, 2001), the occurrence of autoantibodies against OFA/iLRP in human cancer patients has not previously been reported. Although OFA/iLRP is a glycosylated protein (Coggin et al., Arch Otolaryngol Head Neck Surg 119:1257-66, 1993), the data disclosed herein suggested that autoantibodies were not restricted to a glycan containing epitope in OFA/iLRP given the reactivity observed with recombinant LAMR1 in lung cancer patient sera. This observation was consistent with previous findings that bacterially expressed recombinant OFA/iLRP was competent in inducing CTL-mediated target cell lysis (Rohrer et al., J Immunol 176:2844-56, 2006). OFA/iLRP expression was found to precede clear histological evidence of malignant T cells or clinical lymphoma in irradiated mice that went on to develop T-cell lymphomas (Rohrer et al., J Natl Cancer Inst 84:602-9, 1992), consistent with an early immune response during tumorigenesis and the present demonstration of autoantibodies in pre-clinical (e.g., pre-diagnostic) sera.

Although in the present study, no significant difference in PGP9.5 reactivity was observed between pre-diagnostic cases as a group and controls (in contrast to prior findings based on analysis of sera collected at the time of diagnosis of lung cancer), this observation may be related to differences in patient and tumor characteristics or to the temporal pattern of PGP9.5 expression and/or immune response to PGP9.5 in lung cancer. In support of the latter is the finding of increased reactivity among subjects within six months from diagnosis, compared to subjects whose blood was collected 6-12 months prior to diagnosis. Nevertheless, autoantibodies to PGP9.5 may have diagnostic utility in symptomatic subjects in conjunction with an imaging modality.

Disclosed herein for the first time are data related to temporal changes of a humoral immune response to a set of tumor antigens in lung cancer for subjects whose blood was collected over a period ranging from the time of diagnosis to within a year prior to diagnosis as part of the CARET high risk cohort. PGP9.5, 14-3-3 theta and LAMR1 showed increases in reactivity in pre-diagnostic sera with higher reactivity at a time closer to diagnosis (0-6 months) relative to a time farther from diagnosis (7-12 months). Reactivity to annexin I did not show a relationship to time from diagnosis within the one-year time frame. This observation may indicate that an immune response to annexin I occurred earlier during the course of lung cancer development compared to PGP9.5, 14-3-3 theta and LAMR1. It has been demonstrated previously that the sugar moiety on annexin I was important to its antigenicity (Brichory et al., Proc Natl Acad Sci USA 98:9824-9, 2001). This posttranslational modification may have occurred early during tumor development. Alternatively a sugar-containing epitope may have been more immunogenic than a peptide backbone for yielding an early detectable immune response. Tumor antigens with different temporal reactive patterns may have different clinical utility for screening and diagnosis. These findings point to the value of pre-diagnostic sera in assessing the significance of autoreactivity to particular antigens.

While significant reactivity with pre-diagnostic sera was observed in this study for a small panel of antigenic proteins, a screening modality for lung cancer that includes testing for autoantibodies is also contemplated using additional antigenic targets to augment the sensitivity and specificity achieved in this study. An initial application of such a panel may be in conjunction with an imaging screening modality for subjects at an increased risk for lung cancer. Studies aimed at identifying and validating novel antigens are currently being undertaken through the National Cancer Institute Early Detection Research Network (http://edrn.nci.nih.gov) and through other efforts.

TABLE 1
DISTRIBUTION OF LUNG CANCER
HISTOLOGICAL TYPES AMONG PRE-DIAGNOSTIC
LUNG CANCER SERA.

| | Number of subjects | |
|---|---|---|
| Adenocarcinoma | 32 | (38%) |
| Squamous cell carcinoma | 29 | (34%) |
| Other non-small cell lung cancer | 24 | (28%) |
| Total | 85 | |

TABLE 2
TIME FROM BLOOD DRAW TO DIAGNOSIS FOR
PRE-DIAGNOSTIC LUNG CANCER SERA.

| Months | Number of subjects | |
|---|---|---|
| 0-3 | 26 | (31%) |
| 4-6 | 18 | (21%) |
| 7-9 | 19 | (22%) |
| 10-12 | 22 | (26%) |
| Total | 85 | |

TABLE 3
REACTIVITY DIFFERENCES BETWEEN CASES
IN THE 0-6 MONTH GROUP AND THE 7-12
MONTH GROUP.

| | Blood_draw_to_diagnosis | | Mean | |
|---|---|---|---|---|
| Antigens | (month) | n | reactivity | p* |
| Annexin I | 0-6 | 44 | 0.20 | 0.72 |
| | 7-12 | 41 | 0.29 | |
| PGP9.5 | 0-6 | 44 | 0.28 | 0.05 |
| | 7-12 | 41 | −0.11 | |
| LAMR1 | 0-6 | 44 | 0.40 | 0.07 |
| | 7-12 | 41 | −0.05 | |
| 14-3-3 theta | 0-6 | 44 | 0.40 | 0.06 |
| | 7-12 | 41 | −0.03 | |

*Two-sample t test

TABLE 4
REACTIVITY DIFFERENCES BETWEEN CASES
AND CONTROLS IN DIFFERENT GROUPS
STRATIFIED BY BLOOD DRAW TIME TO DIAGNOSIS.

| | Blood_draw_ | | | |
|---|---|---|---|---|
| | to_diagnosis | Mean (SD) | Mean (SD) | |
| Antigens | (month) | cancer | normal | p* |
| Annexin I | 0-6 | 0.20 (1.16) | −0.197 (0.80) | 0.067 |
| | 7-12 | 0.29 (1.25) | −0.293 (0.49) | 0.006 |
| | all | 0.24 (1.20) | −0.244 (0.67) | 0.001 |
| PGP9.5 | 0-6 | 0.28 (1.11) | −0.111 (1.06) | 0.093 |
| | 7-12 | −0.11 (0.64) | −0.078 (1.07) | 0.886 |
| | all | 0.10 (0.93) | −0.095 (1.06) | 0.214 |
| LAMR1 | 0-6 | 0.40 (1.41) | −0.158 (0.49) | 0.017 |
| | 7-12 | −0.05 (0.65) | −0.207 (1.06) | 0.414 |
| | all | 0.18 (1.13) | −0.182 (0.81) | 0.017 |
| 14-3-3 theta | 0-6 | 0.40 (1.13) | −0.254 (0.60) | 0.001 |
| | 7-12 | −0.028 (0.95) | −0.132 (1.13) | 0.654 |
| | all | 0.20 (1.06) | −0.195 (0.89) | 0.010 |

*Two-sample t test

### Example 3

### Exemplary Pre-Diagnostic Lung Cancer Indicator Proteins

ANNEXIN 1
>ipi|IPI00218918|IPI00218918.5 ANNEXIN A1 (SEQ ID NO: 1):
MAMVSEFLKQAWFIENEEQEYVQTVKSSKGGPGSAVSPYPTFN
PSSDVAALHKAIMVKGVDEATIIDILTKRNNAQRQQIKAAYLQETGKPLDETLKK
ALTGHLEEVVLALLKTPAQFDADELRAAMKGLGTDEDTLIEILASRTNKEIRDIN
RVYREELKRDLAKDITSDTSGDFRNALLSLAKGDRSEDFGVNEDLADSDARAL
YEAGERRKGTDVNVFNTILTTRSYPQLRRVFQKYTKYSKHDMNKVLDLELKGD
IEKCLTAIVKCATSKPAFFAEKLHQAMKGVGTRHKALIRIMVSRSEIDMNDIKAF
YQKMYGISLCQAILDETKGDYEKILVALCGGN
>ipi|IPI00549413|IPI00549413.2 ANNEXIN A1 (SEQ ID NO: 2):
MNLILRYTFSKMAMVSEFLKQAWFIENEEQEYVQTVKSSKGGP
GSAVSPYPTFNPSSDVAALHKAIMVKGVDEATIIDILTKRNNAQRQQIKAAYLQE
TGKPLDETLKKALTGHLEEVVLALLKTPAQFDADELRAAMKGLGTDEDTLIEILA
SRTNKEIRDINRVYREELKRDLAKDITSDTSGDFRNALLSLAKGDRSEDFG
>ipi|IPI00643231|IPI00643231.1 ANNEXIN A1 (SEQ ID NO: 3):
MAMVSEFLKQAWFIENEEQEYVQTVKSSKGGPGSAVSPYPTFN
PSSDVAALHKAIMVKGVDEATIIDILTKRNNAQRQQIKAAYLQETGKPLDETLKK
ALTGHLEEVVLALLKTP
14-3-3 THETA
>ipi|IPI00018146|IPI00018146.1 14-3-3 PROTEIN THETA (SEQ ID NO: 4):
MEKTELIQKAKLAEQAERYDDMATCMKAVTEQGAELSNEERNL
LSVAYKNVVGGRRSAWRVISSIEQKTDTSDKKLQLIKDYREKVESELRSICTTV
LELLDKYLIANATNPESKVFYLKMKGDYFRYLAEVACGDDRKQTIDNSQGAYQ
EAFDISKKEMQPTHPIRLGLALNFSVFYYEILNNPELACTLAKTAFDEAIAELDTL
NEDSYKDSTLIMQLLRDNLTLWTSDSAGEECDAAEGAEN
>ipi|IPI00796727|IPI00796727.1 PUTATIVE UNCHARACTERIZED PROTEIN
(SEQ ID NO: 5):
YWHAQMEKTELIQKAKLAEQAERYDDMATCMKAVTEQGAELSN
EERNLLSVAYKNVVGGRRSAWRVISSIEQKTDTSDKKLQLIKDYREKVESELRS
ICTTVLELLDKYLIANATNPESKVFYLKMKGDYFRYLAEVACGDDRKQTIDNSQ
GAY
LAMININ RECEPTOR 1 (LAMR1)
>ipi|IPI00411639|IPI00411639.1 LAMININ RECEPTOR-LIKE
PROTEIN LAMRL5 (SEQ ID NO: 6):
MSGALDVLQMKEEDVLKFLAAGTHLGGTNLDFQMEQYIYKRKS
DGIYIINLKRTWEKLLLTARAIVAIENPADVSVISSRNTGQRAVLKFAAATGATPI
AGRFTPGTFTNQIQAAFREPRLLVVSDPRADHQPLTEASYVNLPTIALCNTDSP
LHYVDIAIPCNNKGTHSVGLMWWMLAREVLRMRGTISREHPWEVMPDLYFYR
DPEEIEKEEQAAAEKAMTREELQGEWTAPAPEFTATQPEVADWSEGVQVPSV
PIQQFPTEDWSTQRATEDWSAAPTAQATEWVGATTDWS
>ipi|IPI00413108|IPI00413108.4 33 KDA PROTEIN (SEQ ID NO: 7):
MSGALDVLQMKEEDVLKFLAAGTHLGGTNLDFQMEQYIYKRKS
DGIYIINLKRTWEKLLLAARAIVAIENPADVSVISSRNTGQVCGTVRAVLKFAAAT
GATPIAGRFTPGTFTNQIQAAFREPRLLWTDPRADHQPLTEASYVNLPTIALC
NTDSPLRYVDIAIPCNNKGAHSVGLMWWMLAREVLRMRGTISREHPWEVMP
DLYFYRDPEEIEKEEQAAAEKAVTKEEFQGEWTAPAPEFTATQPEVADWSEG
VQVPSVPIQQFPTEDWSAQPATEDWSAAPTAQATEWVGATTDWS
>ipi|IPI00553164|IPI00553164.4 40S RIBOSOMAL PROTEIN SA
(SEQ ID NO: 8):
MSGALDVLQMKEEDVLKFLAAGTHLGGTNLDFQMEQYIYKRKS
DGIYIINLKRTWEKLLLAARAIVAIENPADVSVISSRNTGQRAVLKFAAATGATPI
AGRFTPGTFTNQIQAAFREPRLLVVTDPRADHQPLTEASYVNLPTIALCNTDSP
LRYVDIAIPCNNKGAHSVGLMWWMLAREVLRMRGTISREHPWEVMPDLYFYR
DPEEIEKEEQAAAEKAVTKEEFQGEWTAPAPEFTATQPEVADWSEGVQVPSV
PIQQFPTEDWSAQPATEDWSAAPTAQATEWVGATTDWS
>ipi|IPI00790580|IPI00790580.1 16 KDA PROTEIN (SEQ ID NO: 9):
MSGALDVLQMKEEDVLKFLAAGTHLGGTNLDFQMEQYIYKRKS
DGIYIINLKRTWEKLLLAARAIVAIENPADVSVISSRNTGQRAVLKFAAATGATPI
AGRFTPGTFTNQIQAAFREPRLLVVTDPRADHQPLTEASYVNLPTIAL
>ipi|IPI00793905|IPI00793905.1 24 KDA PROTEIN (SEQ ID NO: 10):
MSGALDVLQMKEEDVLKFLAAGTHLGGTNLDFQMEQYIYKRKS
DGIYIINLKRTWEKLLLAARAIVAIENPADVSVISSRNTGQGAHSVGLMWWMLA
REVLRMRGTISREHPWEVMPDLYFYRDPEEIEKEEQAAAEKAVTKEEFQGEW
TAPAPEFTATQPEVADWSEGVQVPSVPIQQFPTEDWSAQPATEDWSAAPTA
QATEWVGATTDWS

### Example 4

### Identification of Additional Pre-Diagnostic Lung Cancer Indicator Proteins

Using the methodologies described above in Examples 1 and 2, additional pre-diagnostic lung cancer indicator proteins were identified on the basis of detection of specifically binding autoantibodies in 30 pre-diagnostic lung cancer sera, using 30 matched normal sera as controls. The results are summarized in Table 5.

TABLE 5
ADDITIONAL PRE-DIAGNOSTIC LUNG CANCER INDICATOR PROTEINS

| | | p-value | sensitivity | | SEQ |
|---|---|---|---|---|---|
| | | for M2 | at 95% | | ID |
| Gene | Uniprot_id | statistics | specificity | Description | NO: |
| AKR1B10 | O60218 | 0.000085 | 0.47 | Aldo-keto reductase family | 11 |
| | | | | 1, member B10 | |
| GOT2 | P00505 | 0.000216 | 0.43 | Glutamic-oxaloacetic | 12 |
| | | | | transaminase 2 | |
| HNRPR | O43390 | 0.001233 | 0.37 | Heterogeneous nuclear | 13 |
| | | | | ribonucleoprotein r | |
| PDIA3 | P30101 | 0.001233 | 0.37 | Protein disulfide-isomerase | 14 |
| | | | | a3 | |
| NME2 | P22392 | 0.001233 | 0.37 | Nonmetastatic cells 2, | 15 |
| | | | | protein expressed in | |
| RTN4 | Q9NQC3 | 0.026157 | 0.23 | Reticulon-4 | 16 |
| HI1FX | Q92522 | 0.026157 | 0.23 | H1 histone family, member x | 17 |
| G3BP | Q13283 | 0.051395 | 0.20 | Ras-GTPase-activating- | 18 |
| | | | | protein SH3-domain Binding | |
| | | | | Protein 1 | |
| HSPCA | P07900 | 0.051395 | 0.20 | Heat shock 90 kda protein 1, | 19 |
| | | | | alpha | |
| ACTN4 | O43707 | 0.051395 | 0.20 | Actinin, alpha 4 | 20 |

SEQ ID NO: 11
MATFVELSTKAKMPIVGLGTWKSPLGKVKEAVKVAIDAGYRHIDCAYVYQNEH
EVGEAIQEKIQEKAVKREDLFIVSKLWPTFFERPLVRKAFEKTLKDLKLSYLDVY
LIHWPQGFKSGDDLFPKDDKGNAIGGKATFLDAWEAMEELVDEGLVKALGVS
NFSHFQIEKLLNKPGLKYKPVTNQVECHPYLTQEKLIQYCHSKGITVTAYSPLG
SPDRPWAKPEDPSLLEDPKIKEIAAKHKKTAAQVLIRFHIQRNVIVIPKSVTPARI
VENIQVFDFKLSDEEMATILSFNRNWRACNVLQSSHLEDYPFDAEY
SEQ ID NO: 12
MALLHSGRVLPGIAAAFHPGLAAAASARASSWWTHVEMGPPDPILGVTEAFK
RDTNSKKMNLGVGAYRDDNGKPYVLPSVRKAEAQIAAKNLDKEYLPIGGLAEF
CKASAELALGENSEVLKSGRFVTVQTISGTGALRIGASFLQRFFKFSRDVFLPK
PTWGNHTPIFRDAGMQLQGYRYYDPKTCGFDFTGAVEDISKIPEQSVLLLHAC
AHNPTGVDPRPEQWKEIATVVKKRNLFAFFDMAYQGFASGDGDKDAWAVRH
FIEQGINVCLCQSYAKNMGLYGERVGAFTMVCKDADEAKRVESQLKILIRPMY
SNPPLNGARIAAAILNTPDLRKQWLQEVKGMADRIIGMRTQLVSNLKKEGSTH
NWQHITDQIGMFCFTGLKPEQVERLIKEFSIYMTKDGRISVAGVTSSNVGYLAH
AIHQVTK
SEQ ID NO: 13
MANQVNGNAVQLKEEEEPMDTSSVTHTEHYKTLIEAGLPQKVAERLDEIFQTG
LVAYVDLDERAIDALREFNEEGALSVLQQFKESDLSHVQNKSAFLCGVMKTYR
QREKQGSKVQESTKGPDEAKIKALLERTGYTLDVTTGQRKYGGPPPDSVYSG
VQPGIGTEVFVGKIPRDLYEDELVPLFEKAGPIWDLRLMMDPLSGQNRGYAFIT
FCGKEAAQEAVKLCDSYEIRPGKHLGVCISVANNRLFVGSIPKNKTKENILEEF
SKVTEGLVDVILYHQPDDKKKNRGFCFLEYEDHKSAAQARRRLMSGKVKVWG
NVVTVEWADPVEEPDPEVMAKVKVLFVRNLATTVTEEILEKSFSEFGKLERVK
KLKDYAFVHFEDRGAAVKAMDEMNGKEIEGEEIEIVLAKPPDKKRKERQAARQ
ASRSTAYEDYYYHPPPRMPPPIRGRGRGGGRGGYGYPPDYYGYEDYYDDYY
GYDYHDYRGGYEDPYYGYDDGYAVRGRGGGRGGRGAPPPPRGRGAPPPR
GRAGYSQRGAPLGPPRGSRGGRGGPAQQQRGRGSRGSRGNRGGNVGGK
RKADGYNQPDSKRRQTNNQQNWGSQPIAQQPLQQGGDYSGNYGYNNDNQ
EFYQDTYGQQWK
SEQ ID NO: 14
MRLRRLALFPGVALLLAAARLAAASDVLELTDDNFESRISDTGSAGLMLVEFFA
PWCGHCKRLAPEYEAAATRLKGIVPLAKVDCTANTNTCNKYGVSGYPTLKIFR
DGEEAGAYDGPRTADGIVSHLKKQAGPASVPLRTEEEFKKFISDKDASIVGFF
DDSFSEAHSEFLKAASNLRDNYRFAHTNVESLVNEYDDNGEGIILFRPSHLTNK
FEDKTVAYTEQKMTSGKIKKFIQENIFGICPHMTEDNKDLIQGKDLLIAYYDVDY
EKNAKGSNYWRNRVMMVAKKFLDAGHKLNFAVASRKTFSHELSDFGLESTA
GEIPVVAIRTAKGEKFVMQEEFSRDGKALERFLQDYFDGNLKRYLKSEPIPESN
DGPVKVVVAENFDEIVNNENKDVLIEFYAPWCGHCKNLEPKYKELGEKLSKDP
NIVIAKMDATANDVPSPYEVRGFPTIYFSPANKKLNPKKYEGGRELSDFISYLQ
REATN PPVIQEEKPKKKKKAQEDL
SEQ ID NO: 15
MANLERTFIAIKPDGVQRGLVGEIIKRFEQKGFRLVAMKFLRASEEHLKQHYIDL
KDRPFFPGLVKYMNSGPVVAMVWEGLNVVKTGRVMLGETNPADSKPGTIRG
DFCIQVGRNIIHGSDSVKSAEKEISLWFKPEELVDYKSCAHDWVYE
SEQ ID NO: 16
MEDLDQSPLVSSSDSPPRPQPAFKYQFVREPEDEEEEEEEEEEDEDEDLEEL
EVLERKPAAGLSAAPVPTAPAAGAPLMDFGNDFVPPAPRGPLPAAPPVAPER
QPSWDPSPVSSTVPAPSPLSAAAVSPSKLPEDDEPPARPPPPPPASVSPQAE
PVWTPPAPAPAAPPSTPAAPKRRGSSGSVDETLFALPAASEPVIRSSAENMDL
KEQPGNTISAGQEDFPSVLLETAASLPSLSPLSAASFKEHEYLGNLSTVLPTEG
TLQENVSEASKEVSEKAKTLLIDRDLTEFSELEYSEMGSSFSVSPKAESAVIVA
NPREEIIVKNKDEEEKLVSNNILHNQQELPTALTKLVKEDEVVSSEKAKDSFNE
KRVAVEAPMREEYADFKPFERVWEVKDSKEDSDMLAAGGKIESNLESKVDKK
CFADSLEQTNHEKDSESSNDDTSFPSTPEGIKDRSGAYITCAPFNPAATESIAT
NIFPLLGDPTSENKTDEKKIEEKKAQIVTEKNTSTKTSNPFLVAAQDSETDYVTT
DNLTKVTEEVVANMPEGLTPDLVQEACESELNEVTGTKIAYETKMDLVQTSEV
MQESLYPAAQLCPSFEESEATPSPVLPDIVMEAPLNSAVPSAGASVIQPSSSPL
EASSVNYESIKHEPENPPPYEEAMSVSLKKVSGIKEEIKEPENINAALQETEAPY
ISIACDLIKETKLSAEPAPDFSDYSEMAKVEQPVPDHSELVEDSSPDSEPVDLF
SDDSIPDVPQKQDETVMLVKESLTETSFESMIEYENKEKLSALPPEGGKPYLES
FKLSLDNTKDTLLPDEVSTLSKKEKIPLQMEELSTAVYSNDDLFISKEAQIRETE
TFSDSSPIEIIDEFPTLISSKTDSFSKLAREYTDLEVSHKSEIANAPDGAGSLPCT
ELPHDLSLKNIQPKVEEKISFSDDFSKNGSATSKVLLLPPDVSALATQAEIESIVK
PKVLVKEAEKKLPSDTEKEDRSPSAIFSAELSKTSVVDLLYWRDIKKTGVVFGA
SLFLLLSLTVFSIVSVTAYIALALLSVTISFRIYKGVIQAIQKSDEGHPFRAYLESE
VAISEELVQKYSNSALGHVNCTIKELRRLFLVDDLVDSLKFAVLMWVFTYVGAL
FNGLTLLILALISLFSVPVIYERHQAQIDHYLGLANKNVKDAMAKIQAKIPGLKRK
AE
SEQ ID NO: 17
MSVELEEALPVTTAEGMAKKVTKAGGSAALSPSKKRKNSKKKNQPGKYSQLV
VETIRRLGERNGSSLAKIYTEAKKVPWFDQQNGRTYLKYSIKALVQNDTLLQVK
GTGANGSFKLNRKKLEGGGERRGAPAAATAPAPTAHKAKKAAPGAAGSRRA
DKKPARGQKPEQRSHKKGAGAKKDKGGKAKKTAAAGGKKVKKAAKPSVPKV
PKGRK
SEQ ID NO: 18
MVMEKPSPLLVGREFVRQYYTLLNQAPDMLHRFYGKNSSYVHGGLDSNGKP
ADAVYGQKEIHRKVMSQNFTNCHTKIRHVDAHATLNDGVVVQVMGLLSNNNQ
ALRRFMQTFVLAPEGSVANKFYVHNDIFRYQDEVFGGFVTEPQEESEEEVEE
PEERQQTPEWPDDSGTFYDQAVVSNDMEEHLEEPVAEPEPDPEPEPEQEP
VSEIQEEKPEPVLEETAPEDAQKSSSPAPADIAQTVQEDLRTFSWASVTSKNL
PPSGAVPVTGIPPHVVKVPASQPRPESKPESQIPPQRPQRDQRVREQRINIPP
QRGPRPIREAGEQGDIEPRRMVRHPDSHQLFIGNLPHEVDKSELKDFFQSYG
NVVELRINSGGKLPNFGFVVFDDSEPVQKVLSNRPIMFRGEVRLNVEEKKTRA
AREGDRRDNRLRGPGGPRGGLGGGMRGPPRGGMVQKPGFGVGRGLAPRQ
SEQ ID NO: 19
MPEETQTQDQPMEEEEVETFAFQAEIAQLMSLIINTFYSNKEIFLRELISNSSDA
LDKIRYESLTDPSKLDSGKELHINLIPNKQDRTLTIVDTGIGMTKADLINNLGTIAK
SGTKAFMEALQAGADISMIGQFGVGFYSAYLVAEKVTVITKHNDDEQYAWESS
AGGSFTVRTDTGEPMGRGTKVILHLKEDQTEYLEERRIKEIVKKHSQFIGYPITL
FVEKERDKEVSDDEAEEKEDKEEEKEKEEKESEDKPEIEDVGSDEEEEKKDG
DKKKKKKIKEKYIDQEELNKTKPIWTRNPDDITNEEYGEFYKSLTNDWEDHLAV
KHFSVEGQLEFRALLFVPRRAPFDLFENRKKKNNIKLYVRRVFIMDNCEELIPE
YLNFIRGVVDSEDLPLNISREMLQQSKILKVIRKNLVKKCLELFTELAEDKENYK
KFYEQFSKNIKLGIHEDSQNRKKLSELLRYYTSASGDEMVSLKDYCTRM KENQ
KHIYYITGETKDQVANSAFVERLRKHGLEVIYMIEPIDEYCVQQLKEFEGKTLVS
VTKEGLELPEDEEEKKKQEEKKTKFENLCKIMKDILEKKVEKVVVSNRLVTSPC
CIVTSTYGWTANMERIMKAQALRDNSTMGYMAAKKHLEINPDHSIIETLRQKAE
ADKNDKSVKDLVILLYETALLSSGFSLEDPQTHANRIYRMIKLGLGIDEDDPTAD
DTSAAVTEEMPPLEGDDDTSRMEEVD
SEQ ID NO: 20
MVDYHAANQSYQYGPSSAGNGAGGGGSMGDYMAQEDDWDRDLLLDPAWE
KQQRKTFTAWCNSHLRKAGTQIENIDEDFRDGLKLMLLLEVISGERLPKPERG
KMRVHKINNVNKALDFIASKGVKLVSIGAEEIVDGNAKMTLGMIWTIILRFAIQDI
SVEETSAKEGLLLWCQRKTAPYKNVNVQNFHISWKDGLAFNALIHRHRPELIE
YDKLRKDDPVTNLNNAFEVAEKYLDIPKMLDAEDIVNTARPDEKAIMTYVSSFY
HAFSGAQKAETAANRICKVLAVNQENEHLMEDYEKLASDLLEWIRRTIPWLED
RVPQKTIQEMQQKLEDFRDYRRVHKPPKVQEKCQLEINFNTLQTKLRLSNRPA
FMPSEGKMVSDINNGWQHLEQAEKGYEEWLLNEIRRLERLDHLAEKFRQKAS
IHEAWTDGKEAMLKHRDYETATLSDIKALIRKHEAFESDLAAHQDRVEQIAAIA
QELNELDYYDSHNVNTRCQKICDQWDALGSLTHSRREALEKTEKQLEAIDQLH
LEYAKRAAPFNNWMESAMEDLQDMFIVHTIEEIEGLISAHDQFKSTLPDADRER
EAILAIHKEAQRIAESNHIKLSGSNPYTTVTPQIINSKWEKVQQLVPKRDHALLE
EQSKQQSNEHLRRQFASQANVVGPWIQTKMEEIGRISIEMNGTLEDQLSHLKQ
YERSIVDYKPNLDLLEQQHQLIQEALIFDNKHTNYTMEHIRVGWEQLLTTIARTI
NEVENQILTRDAKGISQEQMQEFRASFNHFDKDHGGALGPEEFKACLISLGYD
VENDRQGEAEFNRIMSLVDPNHSGLVTFQAFIDFMSRETTDTDTADQVIASFK
VLAGDKNFITAEELRRELPPDQAEYCIARMAPYQGPDAVPGALDYKSFSTALY
GESDL

### REFERENCES

1. Stockert E, Jager E, Chen Y T, et al: A survey of the humoral immune response of cancer patients to a panel of human tumor antigens. J Exp Med 187:1349-54, 1998
2. Tan EM: Autoantibodies as reporters identifying aberrant cellular mechanisms in tumorigenesis. J Clin Invest 108:1411-5, 2001
3. Mintz P J, Kim J, Do K A, et al: Fingerprinting the circulating repertoire of antibodies from cancer patients. Nat Biotechnol 21:57-63, 2003
4. Gourevitch M M, von Mensdorff-Pouilly S, Litvinov S V, et al: Polymorphic epithelial mucin (MUC-1)-containing circulating immune complexes in carcinoma patients. Br J Cancer 72:934-8, 1995
5. Gure A O, Altorki N K, Stockert E, et al: Human lung cancer antigens recognized by autologous antibodies: definition of a novel cDNA derived from the tumor suppressor gene locus on chromosome 3p21.3. Cancer Res 58:1034-41, 1998
6. Yamamoto A, Shimizu E, Ogura T, et al: Detection of auto-antibodies against L-myc oncogene products in sera from lung cancer patients. Int J Cancer 69:283-9, 1996
7. Dunn G P, Bruce A T, Ikeda H, et al: Cancer immunoediting: from immunosurveillance to tumor escape. Nat Immunol 3:991-8, 2002
8. Hanash S: Harnessing immunity for cancer marker discovery. Nat Biotechnol 21:37-8, 2003
9. Old L J, Chen Y T: New paths in human cancer serology. J Exp Med 187:1163-7, 1998
10. Finn O J: Immune response as a biomarker for cancer detection and a lot more. N Engl J Med 353:1288-90, 2005
11. Diesinger I, Bauer C, Brass N, et al: Toward a more complete recognition of immunoreactive antigens in squamous cell lung carcinoma. Int J Cancer 102:372-8, 2002
12. Gure A O, Stocked E, Scanlan M J, et al: Serological identification of embryonic neural proteins as highly immunogenic tumor antigens in small cell lung cancer. Proc Natl Acad Sci USA 97:4198-203, 2000
13. Ali Eldib A M, Ono T, Shimono M, et al: Immunoscreening of a cDNA library from a lung cancer cell line using autologous patient serum: Identification of XAGE-1b as a dominant antigen and its immunogenicity in lung adenocarcinoma. Int J Cancer 108:558-63, 2004
14. Yang F, Xiao Z Q, Zhang X Z, et al: Identification of tumor antigens in human lung squamous carcinoma by serological proteome analysis. J Proteome Res 6:751-8, 2007
15. Hanash S: Disease proteomics. Nature 422:226-32, 2003
16. Imafuku Y, Omenn G S, Hanash S: Proteomics approaches to identify tumor antigen directed autoantibodies as cancer biomarkers. Dis Markers 20:149-53, 2004
17. Brichory F, Beer D, Le Naour F, et al: Proteomics-based identification of protein gene product 9.5 as a tumor antigen that induces a humoral immune response in lung cancer. Cancer Res 61:7908-12, 2001
18. Brichory F M, Misek D E, Yim A M, et al: An immune response manifested by the common occurrence of annexins I and II autoantibodies and high circulating levels of IL-6 in lung cancer. Proc Natl Acad Sci USA 98:9824-9, 2001
19. Pereira-Faca S R, Kuick R, Purays E, et al: Identification of 14-3-3 theta as an antigen that induces a humoral response in lung cancer. Cancer Res 67:12000-6, 2007
20. Wang H, Hanash S: Multi-dimensional liquid phase based separations in proteomics. J Chromatogr B Analyt Technol Biomed Life Sci 787:11-8, 2003
21. Faca V, Pitteri S J, Newcomb L, et al: Contribution of protein fractionation to depth of analysis of the serum and plasma proteomes. J Proteome Res 6:3558-65, 2007
22. Nam M J, Madoz-Gurpide J, Wang H, et al: Molecular profiling of the immune response in colon cancer using protein microarrays: occurrence of autoantibodies to ubiquitin C-terminal hydrolase L3. Proteomics 3:2108-15, 2003
23. Madoz-Gurpide J, Kuick R, Wang H, et al: Integral protein microarrays for the identification of lung cancer antigens in sera that induce a humoral immune response. Mol Cell Proteomics, 2007
24. Forrester S, Qiu J, L. M, et al: An experimental strategy for quantitative analysis of the humoral immune response to prostate cancer antigens using natural protein microarrays. Proteomics—Clinical Applications 1:494-505, 2007
25. Goodman G E, Thornquist M D, Balmes J, et al: The Beta-Carotene and Retinol Efficacy Trial: incidence of lung cancer and cardiovascular disease mortality during 6-year follow-up after stopping beta-carotene and retinol supplements. J Natl Cancer Inst 96:1743-50, 2004
26. Omenn G S, Goodman G E, Thornquist M D, et al: Effects of a combination of beta carotene and vitamin A on lung cancer and cardiovascular disease. N Engl J Med 334:1150-5, 1996
27. Qiu J, Madoz-Gurpide J, Misek D E, et al: Development of natural protein microarrays for diagnosing cancer based on an antibody response to tumor antigens. J Proteome Res 3:261-7, 2004
28. Friedman J, Hastie T, Tibshirani R: Additive Logistic Regression: A Statistical View of Boosting. Annals of Statistics 28:337-407, 2000
29. Yasui Y, Pepe M, Thompson M L, et al: A data-analytic strategy for protein biomarker discovery: profiling of high-dimensional proteomic data for cancer detection. Biostatistics 4:449-63, 2003
30. Madoz-Gurpide J, Wang H, Misek D E, et al: Protein based microarrays: a tool for probing the proteome of cancer cells and tissues. Proteomics 1:1279-87, 2001
31. He P, Naka T, Serada S, et al: Proteomics-based identification of alpha-enolase as a tumor antigen in non-small lung cancer. Cancer Sci 98:1234-40, 2007
32. Tureci O, Mack U, Luxemburger U, et al: Humoral immune responses of lung cancer patients against tumor antigen NY-ESO-1. Cancer Lett 236:64-71, 2006
33. Chang J W, Lee S H, Jeong J Y, et al: Peroxiredoxin-I is an autoimmunogenic tumor antigen in non-small cell lung cancer. FEBS Lett 579:2873-7, 2005
34. Yagihashi A, Asanuma K, Kobayashi D, et al: Detection of autoantibodies to livin and survivin in Sera from lung cancer patients. Lung Cancer 48:217-21, 2005
35. Matsumoto S, Teramoto H, Nakamoto M, et al: Presence of antibodies against retinoblastoma tumor suppressor protein in patients with lung cancer. Int J Oncol 19:1035-9, 2001
36. Fernandez-Madrid F, VandeVord P J, Yang X, et al: Antinuclear antibodies as potential markers of lung cancer. Clin Cancer Res 5:1393-400, 1999
37. Lubin R, Zalcman G, Bouchet L, et al: Serum p53 antibodies as early markers of lung cancer. Nat Med 1:701-2, 1995
38. Yow H K, Wong J M, Chen H S, et al: Increased mRNA expression of a laminin-binding protein in human colon carcinoma: complete sequence of a full-length cDNA encoding the protein. Proc Natl Acad Sci USA 85:6394-8, 1988
39. Buto S, Tagliabue E, Ardini E, et al: Formation of the 67-kDa laminin receptor by acylation of the precursor. J Cell Biochem 69:244-51, 1998
40. Rao C N, Castronovo V, Schmitt M C, et al: Evidence for a precursor of the high-affinity metastasis-associated murine laminin receptor. Biochemistry 28:7476-86, 1989
41. Lesot H, Kuhl U, Mark K V: Isolation of a laminin-binding protein from muscle cell membranes. Embo J 2:861-865, 1983
42. Malinoff H L, Wicha M S: Isolation of a cell surface receptor protein for laminin from murine fibrosarcoma cells. J Cell Biol 96:1475-9, 1983
43. Auth D, Brawerman G: A 33-kDa polypeptide with homology to the laminin receptor: component of translation machinery. Proc Natl Acad Sci USA 89:4368-72, 1992
44. Ardini E, Pesole G, Tagliabue E, et al: The 67-kDa laminin receptor originated from a ribosomal protein that acquired a dual function during evolution. Mol Biol Evol 15:1017-25, 1998
45. Coggin J H, Jr., Barsoum A L, Rohrer J W: 37 kiloDalton oncofetal antigen protein and immature laminin receptor protein are identical, universal T-cell inducing immunogens on primary rodent and human cancers. Anticancer Res 19:5535-42, 1999
46. Coggin J H, Jr., Barsoum A L, Rohrer J W: Tumors express both unique TSTA and crossprotective 44 kDa oncofetal antigen. Immunol Today 19:405-8, 1998
47. Rieger R, Edenhofer F, Lasmezas C I, et al: The human 37-kDa laminin receptor precursor interacts with the prion protein in eukaryotic cells. Nat Med 3:1383-8, 1997
48. Wewer U M, Taraboletti G, Sobel M E, et al: Role of laminin receptor in tumor cell migration. Cancer Res 47:5691-8, 1987
49. Menard S, Tagliabue E, Colnaghi M I: The 67 kDa laminin receptor as a prognostic factor in human cancer. Breast Cancer Res Treat 52:137-45, 1998
50. Menard S, Castronovo V, Tagliabue E, et al: New insights into the metastasis-associated 67 kD laminin receptor. J Cell Biochem 67:155-65, 1997
51. Cioce V, Castronovo V, Shmookler B M, et al: Increased expression of the laminin receptor in human colon cancer. J Natl Cancer Inst 83:29-36, 1991
52. Fontanini G, Vignati S, Chine S, et al: 67-Kilodalton laminin receptor expression correlates with worse prognostic indicators in non-small cell lung carcinomas. Clin Cancer Res 3:227-31, 1997
53. Ambrose K R, Anderson N G, Coggin J H: Interruption of SV40 oncogenesis with human foetal antigen. Nature 233:194-5, 1971
54. Coggin J H, Jr., Ambrose K R, Bellomy B B, et al: Tumor immunity in hamsters immunized with fetal tissues. J Immunol 107:526-33, 1971
55. Ambrose K R, Anderson N G, Coggin J H, Jr.: Cytostatic antibody and SV40 tumour immunity in hamsters. Nature 233:321-4, 1971
56. Payne W J, Jr., Coggin J H, Jr.: Mouse monoclonal antibody to embryonic antigen: development, cross-reactivity with rodent and human tumors, and preliminary polypeptide characterization. J Natl Cancer Inst 75:527-44, 1985
57. Coggin J H, Jr., Rohrer S D, Leinbach E D, et al: Radiation-induced lymphoblastic lymphomas/leukemias and sarcomas of mice express conserved, immunogenic 44-kilodalton oncofetal antigen. Am J Pathol 130:136-46, 1988
58. Gussack G S, Rohrer S D, Hester R B, et al: Human squamous cell carcinoma lines express oncofetal 44-kD polypeptide defined by monoclonal antibody to mouse fetus. Cancer 62:283-90, 1988
59. Rohrer J W, Rohrer S D, Barsoum A, et al: Differential recognition of murine tumor-associated oncofetal transplantation antigen and individually specific tumor transplantation antigens by syngeneic cloned BALB/c and RFM mouse T cells. J Immunol 152:754-64, 1994
60. Siegel S, Wagner A, Friedrichs B, et al: Identification of HLA-A*0201-presented T cell epitopes derived from the oncofetal antigen-immature laminin receptor protein in patients with hematological malignancies. J Immunol 176:6935-44, 2006
61. Rohrer J W, Culpepper C, Barsoum A L, et al: Characterization of RFM mouse T lymphocyte anti-oncofetal antigen immunity in apparent tumor-free, long-term survivors of sublethal X-irradiation by limiting dilution T lymphocyte cloning. J Immunol 154:2266-80, 1995
62. Holtl L, Zelle-Rieser C, Gander H, et al: Immunotherapy of metastatic renal cell carcinoma with tumor lysate-pulsed autologous dendritic cells. Clin Cancer Res 8:3369-76, 2002
63. Rohrer J W, Coggin J H, Jr.: CD8 T cell clones inhibit antitumor T cell function by secreting IL-10. J Immunol 155:5719-27, 1995
64. Rohrer J W, Barsoum A L, Coggin J H, Jr.: Identification of oncofetal antigen/immature laminin receptor protein epitopes that activate BALB/c mouse OFA/iLRP-specific effector and regulatory T cell clones. J Immunol 176:2844-56, 2006
65. Rohrer J W, Barsoum A L, Dyess D L, et al: Human breast carcinoma patients develop clonable oncofetal antigen-specific effector and regulatory T lymphocytes. J Immunol 162:6880-92, 1999
66. Rohrer J, Barsoum A, Coggin J: The Development of a New Universal Tumor Rejection Antigen Expressed on Human and Rodent Cancers for Vacccination, Prevention of Cancer, and Anti-Tumor Therapy. Mod Asp Immunobiol 1:191-5, 2001
67. Coggin J H, Jr., Rohrer S D, Hester R D, et al: 44-kd oncofetal transplantation antigen in rodent and human fetal cells. Implications of recrudescence in human and rodent cancers. Arch Otolaryngol Head Neck Surg 119:1257-66, 1993
68. Rohrer S D, Sarli R N, Barsoum A L, et al: Expression of 44-kilodalton oncofetal antigen as a premalignancy marker in X irradiation-induced murine T-cell lymphoma. J Natl Cancer Inst 84:602-9, 1992

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for diagnosing lung cancer in a pre-diagnostic subject, comprising:
(a) contacting (i) one or more antibodies from a biological fluid from the pre-diagnostic subject, and (ii) at least one isolated pre-diagnostic lung cancer indicator protein, under conditions and for a time sufficient for detecting specific binding of at least one antibody from the biological fluid to one or more of said pre-diagnostic lung cancer indicator proteins, and therefrom identifying presence of lung cancer in the pre-diagnostic subject.

2. A screening method for lung cancer, comprising:
(a) contacting (i) one or more antibodies from a biological fluid from each subject of one or a plurality of subjects, and (ii) at least one isolated pre-diagnostic lung cancer indicator protein, under conditions and for a time sufficient for detecting specific binding of at least one antibody from the biological fluid to one or more of said pre-diagnostic lung cancer indicator proteins, wherein detection of specific binding indicates the subject has lung cancer, and thereby screening for lung cancer.

3. A method for diagnosing lung cancer in a pre-diagnostic subject, comprising:
(a) contacting (i) one or more antibodies from a biological fluid from the pre-diagnostic subject, and (ii) an isolated protein or polypeptide that comprises one or more antigenic epitopes of one or more pre-diagnostic lung cancer indicator proteins, under conditions and for a time sufficient for detecting specific binding of at least one antibody from the biological fluid to one or more of said antigenic epitopes, and therefrom identifying presence of lung cancer in the pre-diagnostic subject.

4. A screening method for lung cancer, comprising:
(a) contacting (i) one or more antibodies from a biological fluid from each subject of one or a plurality of subjects, and (ii) an isolated protein or polypeptide that comprises one or more antigenic epitopes of one or more pre-diagnostic lung cancer indicator proteins, under conditions and for a time sufficient for detecting specific binding of at least one antibody from the biological fluid to one or more of said antigenic epitopes, wherein detection of specific binding indicates the subject has lung cancer, and thereby screening for lung cancer.

5. The method of claim 1 wherein at least one of the one or more pre-diagnostic lung cancer indicator proteins comprises a LAMR1 protein.

6. The method of claim 5 wherein the pre-diagnostic lung cancer indicator proteins further comprise at least one protein selected from the group consisting of:
(a) annexin I protein
(b) 14-3-3 theta protein

7. The method of claim 1 wherein the lung cancer is selected from the group consisting of (i) adenocarcinoma, (ii) squamous cell carcinoma, (iii) non-small cell lung cancer that is not (i) or (ii), and (iv) lung cancer that can be defined based on one or more of causation and gene mutational status.

8. The method of claim 6 wherein the lung cancer is selected from the group consisting of (i) adenocarcinoma, (ii) squamous cell carcinoma, (iii) non-small cell lung cancer that is not (i) or (ii), and (iv) lung cancer that can be defined based on one or more of causation and gene mutational status.

9. The method of claim 1 wherein the subject or pre-diagnostic subject is at increased risk for developing lung cancer.

10. The method of claim 9 wherein the subject or pre-diagnostic subject has at least one indicator of increased risk for developing lung cancer that is selected from the group consisting of (i) a history of asbestos exposure, (ii) a history of smoking tobacco products, (iii) a history of radon gas exposure, (iv) a history of exposure to a source of ionizing radiation, (v) a history of recurrent lung inflammation, (vi) a history of tuberculosis, (vi) a history of silicosis, berylliosis or talc inhalation, (vii) a family history of lung cancer in genetically related individuals, (viii) a history of vitamin A deficiency or vitamin A excess, (ix) a history of smoking cannabis, and (x) exposure to toxic volatile substances or infectious agents.

11. The method of claim 1 wherein the antibodies are isolated from the biological fluid prior to the step of contacting.

12. The method of claim 1 wherein the antibodies are present in the biological fluid during the step of contacting.

13. The method of claim 1 wherein the antibodies are autoantibodies.

14. The method of claim 1 wherein the biological fluid is selected from the group consisting of blood, serum, serosal fluid, plasma, lymph, urine, cerebrospinal fluid, saliva, a mucosal secretion, a vaginal secretion, ascites fluid, pleural fluid, pericardial fluid, peritoneal fluid, abdominal fluid, culture medium, conditioned culture medium and lavage fluid.

15. The method of claim 1 wherein the biological fluid comprises serum.

16. The method of claim 1 wherein the pre-diagnostic indicator protein, or the isolated protein or polypeptide that comprises one or more antigenic epitopes of a pre-diagnostic indicator protein, is selected from the group consisting of:
(i) a naturally occurring protein or polypeptide,
(ii) a synthetic protein or polypeptide,
(iii) a recombinant protein or polypeptide, and
(iv) a fusion protein or polypeptide that comprises a fusion polypeptide domain fused to the pre-diagnostic indicator protein, or to the polypeptide that comprises one or more antigenic epitopes of the pre-diagnostic indicator protein.

17. The method of claim 1 wherein the pre-diagnostic indicator protein, or the isolated protein or polypeptide that comprises one or more antigenic epitopes of a pre-diagnostic indicator protein, is immobilized on a solid substrate.

18. The method of claim 17 wherein the immobilized pre-diagnostic indicator protein or the immobilized isolated protein or polypeptide that comprises one or more antigenic epitopes of a pre-diagnostic indicator protein, is immobilized by a covalent bond.

19. The method of claim 17 wherein the immobilized pre-diagnostic indicator protein or the immobilized isolated protein or polypeptide that comprises one or more antigenic epitopes of a pre-diagnostic indicator protein, is non-covalently immobilized.

20. The method of claim 1 wherein detecting specific binding of the at least one antibody comprises detecting a signal that is selected from the group consisting of a fluorescent signal, a radiometric signal, an enzymatic signal and a spectrometric signal.

21. The method of claim 1 wherein the pre-diagnostic lung cancer indicator protein is selected from the group consisting of (i) a non-posttranslationally modified protein, (ii) a posttranslationally modified protein that is selected from a glycoprotein, a lipoprotein, a phosphoprotein, a proteolipid, a glypiated protein, a ubiquitinylated protein, a SUMOylated protein, a sulfated protein and a glycated protein, and (iii) a posttranslationally modified protein of (ii) in which one or more posttranslational modifications result in immunogenicity.

22. The method of claim 1 wherein at least one of the one or more pre-diagnostic lung cancer indicator proteins comprises a protein that is selected from the group consisting of (a) AKR1B10 protein [SEQ ID NO:11], (b) GOT2 protein [SEQ ID NO:12], (c) HNRPR protein [SEQ ID NO:13], (d) PDIA3 protein [SEQ ID NO:14], (e) NME2 protein [SEQ ID NO:15], (f) RTN4 protein [SEQ ID NO:16], (g) HI1FX protein [SEQ ID NO:17], (h) G3BP protein [SEQ ID NO:18], (i) HSPCA protein [SEQ ID NO:19], and (j) ACTN4 protein [SEQ ID NO:20].

23. The method of claim 5 wherein the pre-diagnostic lung cancer indicator proteins further comprise at least one protein that is selected from the group consisting of (a) AKR1B10 protein [SEQ ID NO:11], (b) GOT2 protein [SEQ ID NO:12], (c) HNRPR protein [SEQ ID NO:13], (d) PDIA3 protein [SEQ ID NO:14], (e) NME2 protein [SEQ ID NO:15], (f) RTN4 protein [SEQ ID NO:16], (g) HI1FX protein [SEQ ID NO:17], (h) G3BP protein [SEQ ID NO:18], (i) HSPCA protein [SEQ ID NO:19], and (j) ACTN4 protein [SEQ ID NO:20].

24. A method of monitoring lung cancer autoimmune reactivity in a lung cancer patient, comprising:
(a) contacting, after each of two or more timepoints,
(i) one or more antibodies from a biological fluid that is taken from a subject at each of said timepoints, and
(ii) a test antigen that is selected from the group consisting of (1) at least one isolated pre-diagnostic lung cancer indicator protein and (2) at least one isolated protein or polypeptide that comprises one or more antigenic epitopes of one or more pre-diagnostic lung cancer indicator proteins, under conditions and for a time sufficient for detecting specific binding of at least one antibody from the biological fluid to one or more of said pre-diagnostic lung cancer indicator proteins or antigenic epitopes thereof; and
(b) comparing the specific binding that is detectable by antibodies from the biological fluid taken at each of said two or more timepoints, and thereby monitoring lung cancer autoimmune reactivity in the patient.

25. The method of claim 24 wherein a first timepoint occurs before administration of a therapeutic agent to the patient and a second timepoint occurs after administration of the therapeutic agent to the patient.
